(19) United States
(12) Patent Application Publication
(10) Pub. No.: US 2017/0205619 A1
(43) Pub. Date: Jul. 20, 2017

(54) ENDOSCOPE SYSTEM

(71) Applicant: OLYMPUS CORPORATION, Tokyo (JP)

(72) Inventors: Toshihiro HAMADA, Tokyo (JP); Takeo SUZUKI, Tokyo (JP)

(73) Assignee: OLYMPUS CORPORATION, Tokyo (JP)

(21) Appl. No.: 15/479,765

(22) Filed: Apr. 5, 2017

Related U.S. Application Data
(63) Continuation of application No. PCT/JP2015/085976, filed on Dec. 24, 2015.

(30) Foreign Application Priority Data
Jan. 5, 2015 (JP) 2015-000448

(51) Int. Cl. G02B 23/24; H04N 5/225; A61B 1/06; A61B 1/005; A61B 1/04; A61B 1/00

(52) U.S. Cl. CPC G02B 23/2484; A61B 1/04; A61B 1/00045; A61B 1/06; A61B 1/0051; H04N 5/2258; H04N 2005/2255

(57) ABSTRACT

An endoscope system includes: an insertion portion configured to be inserted into an inside of an object; a forward observation window configured to acquire a forward field-of-view image; a lateral observation window configured to acquire a lateral field-of-view image; and a control portion. The control portion detects an amount of change in an image signal in a predetermined judgment area in at least one of the forward field-of-view image and the lateral field-of-view image within a predetermined time period, judges a use state of the insertion portion based on a result of the detection, and controls an image signal of the forward field-of-view image and an image signal of the lateral field-of-view image to be outputted to a monitor capable of displaying the forward field-of-view image and the lateral field-of-view image, according to the judged use state.

### CROSS REFERENCE TO RELATED APPLICATION

This application is a continuation application of PCT/JP2015/085976 filed on Dec. 24, 2015 and claims benefit of Japanese Application No. 2015-000448 filed in Japan on Jan. 5, 2015, the entire contents of which are incorporated herein by this reference.

### BACKGROUND OF THE INVENTION

1. Field of the Invention

The present invention relates to an endoscope system, and in particular to an endoscope system capable of observing forward and lateral directions simultaneously.

2. Description of the Related Art

Endoscope systems provided with an endoscope configured to pick up an image of an object inside a subject, an image processing apparatus configured to generate an observation image of the object which has been image-picked up by the endoscope and the like are widely used in a medical field, an industrial field and the like. A user of such an endoscope system, for example, an operator can insert an insertion portion of the endoscope into a subject to perform observation of an inside of the subject, treatment and the like.

Further, some endoscope systems are capable of observing a subject with a wide field of view for purposes of prevention of oversight of a lesion and the like. In the case of a wide field-of-view endoscope, however, an amount of information included in an image is relatively large in comparison with a conventional endoscope, and, therefore, there is a problem that an area of interest is displayed relatively small.

Therefore, Japanese Patent Application Laid-Open Publication No. 2012-245157 proposes an endoscope apparatus which sets an area of interest in a wide-angle endoscopic image and performs a process for locally changing a magnification to enlarge the area of interest to be relatively larger than other areas, in order to display the area of interest in an appropriate size.

Furthermore, Japanese Patent Application Laid-Open Publication No. 1111-32982 proposes an endoscope apparatus capable of displaying a front-view image and a side-view image and capable of changing a form of displaying only the front-view image to a form of displaying both of the front-view image and the side-view image and changing the form of displaying both of the front-view image and the side-view image to a form of displaying the front-view image by enlarging the front-view image to be larger than the side-view image.

### SUMMARY OF THE INVENTION

An endoscope system of an aspect of the present invention includes: an insertion portion configured to be inserted into an inside of an object; a first object image acquiring portion provided on the insertion portion and configured to acquire a first object image from a first area of the object; a second object image acquiring portion provided on the insertion portion and configured to acquire a second object image from a second area of the object different from the first area; an image change amount detecting portion configured to detect an amount of change in an image signal in a predetermined area of at least one of the first object image and the second object image within a predetermined time period; and an image signal generating portion configured to generate an image signal based on the first object image and generate an image signal obtained by changing a display information amount of the second object image according to the amount of the change in the image signal detected by the image change amount detecting portion.

### BRIEF DESCRIPTION OF THE DRAWINGS

FIG. 1 is a diagram showing a configuration of an endoscope system 1 according to a first embodiment of the present invention;

FIG. 2 is a perspective view showing a configuration of a distal end portion 6 of an insertion portion 4 of an endoscope 2 according to the first embodiment of the present invention;

FIG. 3 is a front view showing the configuration of the distal end portion 6 of the insertion portion 4 of the endoscope 2 according to the first embodiment of the present invention;

FIG. 4 is a diagram showing an example of an observation image displayed on a monitor 35 by image processing by a video processor 32 of the endoscope system 1 according to the first embodiment of the present invention;

FIG. 5 is a block diagram showing a configuration of the video processor 32 according to the first embodiment of the present invention;

FIG. 6 is a flowchart showing an example of a flow of a whole process of a control portion 45 in an automatic image display switching mode in the endoscope system 1 of the first embodiment of the present invention;

FIG. 7 is a flowchart showing an example of a flow of a judgment area setting process in an initial setting process (S1) according to the first embodiment of the present invention;

FIG. 8 is a flowchart showing an example of a flow of a mask area setting process in the initial setting process (S1) according to the first embodiment of the present invention;

FIG. 9 is a flowchart showing an example of a flow of an image change amount detection process (S2) according to the first embodiment of the present invention;

FIG. 10 is a diagram for illustrating a judgment area set in an endoscopic image displayed on the monitor 35 according to the first embodiment of the present invention;

FIG. 11 is a diagram showing an example of an observation image displayed on the monitor 35 in an insertion state, according to the first embodiment of the present invention;

FIG. 12 is a diagram showing an example of an observation image when a treatment instrument appears in the observation image at time of screening, according to the first embodiment of the present invention;

FIG. 13 is a diagram showing an example of an observation image displayed on the monitor 35 in a treatment instrument used state, according to the first embodiment of the present invention;

FIG. 14 is a diagram showing an example of the observation image displayed on the monitor 35 in the treatment instrument used state, according to the first embodiment of the present invention;

FIG. 15 is a schematic diagram showing a configuration of a distal end portion 6 of an endoscope 2A of a second embodiment of the present invention;

FIG. 16 is a block diagram showing a configuration of a video processor 32A according to the second embodiment of the present invention;

FIG. 17 is a diagram showing a display example of three endoscopic images displayed on three monitors 35A, 35B and 35C, according to the second embodiment of the present invention;

FIG. 18 is a diagram showing a display example of three endoscopic images displayed on one monitor 35, according to the second embodiment of the present invention;

FIG. 19 is a diagram showing an example of observation images displayed on the three monitors 35A, 35B and 35C when an endoscope system 1A is set to an automatic image display switching mode, according to the second embodiment of the present invention;

FIG. 20 is a diagram showing an example of observation images displayed on the three monitors 35A, 35B and 35C in an insertion state, according to the second embodiment of the present invention;

FIG. 21 is a diagram showing an example of observation images displayed on the three monitors 35A, 35B and 35C in a treatment instrument used state, according to the second embodiment of the present invention;

FIG. 22 is a diagram showing an example of an observation image displayed on the monitor 35A by enlarging an image area which includes a treatment instrument MI in a lateral field-of-view image SV1, according to the second embodiment of the present invention; and

FIG. 23 is a perspective view of a distal end portion 6a of an insertion portion 4 to which a unit for lateral observation is attached, according to a modification of the second embodiment of the present invention.

### DETAILED DESCRIPTION OF THE PREFERRED EMBODIMENT(S)

Embodiments of the present invention will be described below with reference to drawings.

### First Embodiment

(System configuration)

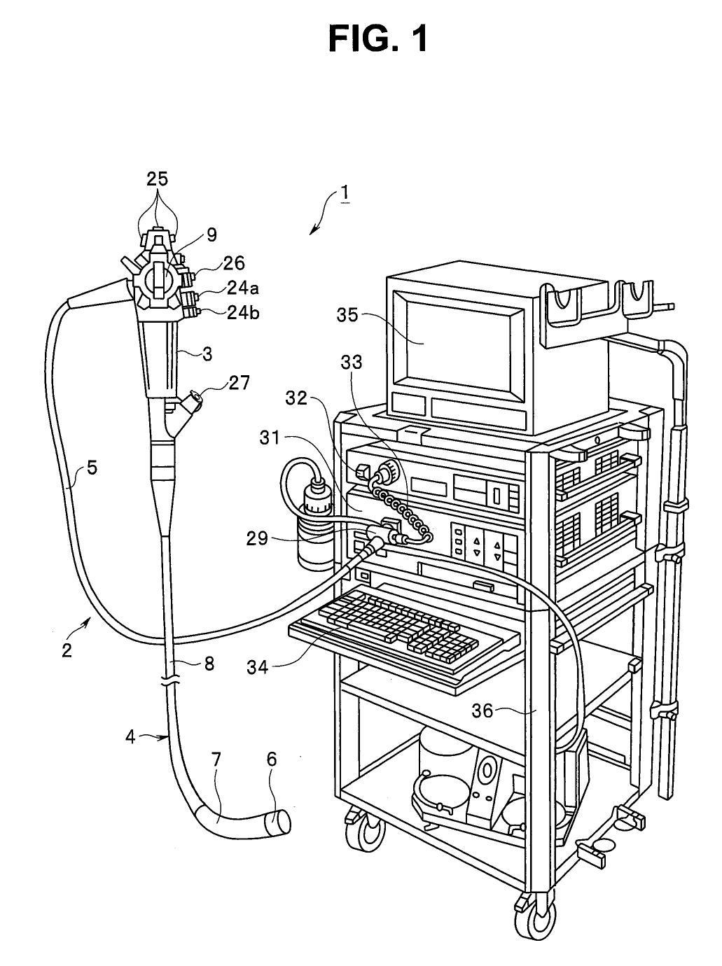

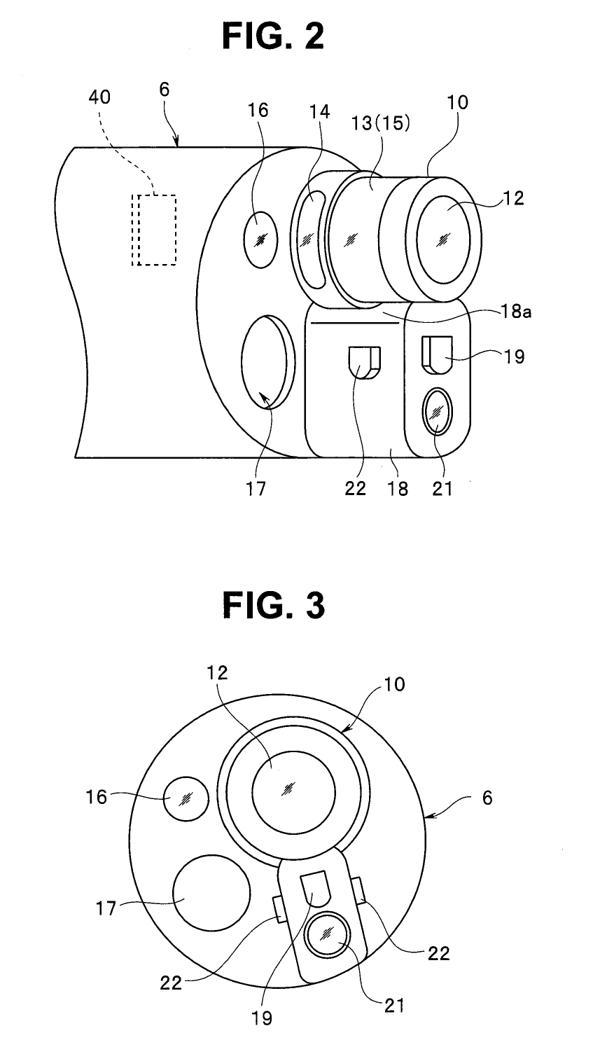

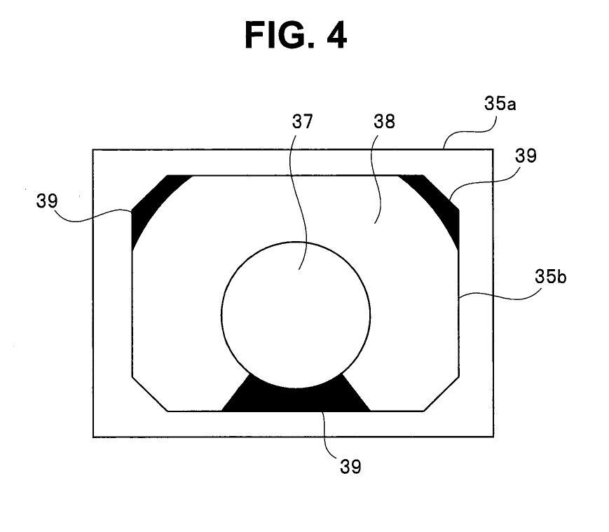

First, a configuration of an endoscope system 1 of a first embodiment will be described with use of FIGS. 1 to 4. FIG. 1 is a diagram showing the configuration of the endoscope system 1 according to the first embodiment of the present invention; FIG. 2 is a perspective view showing a configuration of a distal end portion 6 of an insertion portion 4 of an endoscope 2; FIG. 3 is a front view showing the configuration of the distal end portion 6 of the insertion portion 4 of the endoscope 2; and FIG. 4 is a diagram showing an example of an observation image displayed on a monitor 35 as a display portion by image processing by a video processor 32 of the endoscope system 1.

As shown in FIG. 1, the endoscope system 1 has the endoscope 2 configured to pick up an image of an observation target object (an object) and output an image pickup signal; a light source apparatus 31 configured to supply illumination light for illuminating the observation target object; the video processor 32 which is an image processing apparatus configured to generate and output a video signal corresponding to the image pickup signal; and the monitor 35 configured to display an observation image which is an endoscopic image corresponding to the video signal.

The endoscope 2 is configured having an operation portion 3 for an operator to grasp to perform an operation; the elongated insertion portion 4 formed on a distal end side of the operation portion 3 and configured to be inserted into a body cavity or the like, which is an object; and a universal cord 5 one end portion of which is provided so as to extend from a side portion of the operation portion 3.

The endoscope 2 of the present embodiment is a wide-angle endoscope making it possible to observe a field of view with an angle of 180 degrees or more by causing a plurality of field-of-view images to be displayed, and realizes prevention of oversight of a lesion at a place where it is difficult to find the lesion only by forward observation, such as a back of a fold and an organ boundary, in a body cavity, especially in a large intestine. At time of inserting the insertion portion 4 of the endoscope 2 into a large intestine, an operation of temporary fixation and the like is generated by causing the insertion portion 4 to be twisted, to perform a reciprocating motion, and to be hooked on an intestinal wall.

The insertion portion 4 to be inserted into an inside of an object is configured having the rigid distal end portion 6 provided on a most distal end side, a bending portion 7 capable of freely bending which is provided on a rear end of the distal end portion 6, and a long flexible tube portion 8 having flexibility, which is provided on a rear end of the bending portion 7. Further, the bending portion 7 performs a bending operation corresponding to an operation of a bending operation lever 9 provided on the operation portion 3.

On the other hand, as shown in FIG. 2, a columnar-shaped cylindrical portion 10 provided projecting from a position displaced upward from a center of a distal end face of the distal end portion 6 is formed on the distal end portion 6 of the insertion portion 4.

An objective optical system for observation of both of a forward field of view and a lateral field of view, which is not shown, is provided on a distal end portion of the cylindrical portion 10. Further, the distal end portion of the cylindrical portion 10 is configured having a forward observation window 12 arranged at a position corresponding to a forward direction of the objective optical system not shown, and a lateral observation window 13 arranged at a position corresponding to a side-view direction of the objective optical system not shown. Furthermore, a lateral direction illuminating portion 14 configured to emit light for illuminating a lateral direction is formed near the proximal end of the cylindrical portion 10. The lateral observation window 13 is arranged on a proximal end side of the insertion portion 4 with respect to the forward observation window 12.

The lateral observation window 13 is provided with a side-view mirror lens 15 for making it possible to acquire a lateral field-of-view image by catching return light from an observation target object caused to incident from around the columnar-shaped cylindrical portion 10, that is, reflected light within the lateral field of view.

Note that an image pickup surface of an image pickup device 40 is arranged at an image forming position of the objective optical system not shown so that an image of the observation target object within a field of view of the forward observation window 12 is formed in a central part as a circular forward field-of-view image, and an image of the observation target object within a field of view of the lateral observation window 13 is formed in an outer circumferential part of the forward field-of-view image as an annular shaped lateral field-of-view image.

The forward observation window 12 is provided on the distal end portion 6 in a longitudinal direction of the insertion portion 4 and constitutes a first image acquiring portion configured to acquire a first object image from a first area which includes a direction in which the insertion portion 4 is inserted (the forward direction) which is a first direction. In other words, the forward observation window 12 is a forward image acquiring portion configured to acquire an object image of an area which includes a forward direction of the insertion portion 4, and the first object image is an object image of an area which includes the forward direction of the insertion portion 4 almost parallel to the longitudinal direction of the insertion portion 4.

The lateral observation window 13 is provided on the distal end portion 6 in the longitudinal direction of the insertion portion 4 and constitutes a second image acquiring portion configured to acquire a second object image from a second area which includes a lateral direction of the insertion portion 4 which is a second direction different from the first direction. In other words, the lateral observation window 13 is a lateral image acquiring portion configured to acquire an object image of an area which includes a direction crossing the longitudinal direction of the insertion portion 4, for example, at right angles, and the second object image is an object image of an area which includes the lateral direction of the insertion portion 4 which is a direction crossing the longitudinal direction of the insertion portion 4.

The image pickup device 40, which is an image pickup portion, photoelectrically converts the forward field-of-view image and the lateral field-of-view image on one image pickup surface, and an image signal of the forward field-of-view image and an image signal of the lateral field-of-view image are generated by being cut out from images obtained by the image pickup device 40.

On the distal end face of the distal end portion 6, a forward illumination window 16 arranged at a position adjoining the cylindrical portion 10 and configured to emit illumination light within a range of a forward field of view of the forward observation window 12, and a distal end opening portion 17 communicating with a treatment instrument channel not shown, which is formed with a tube or the like arranged in the insertion portion 4, and being capable of causing a distal end portion of a treatment instrument inserted in the treatment instrument channel to project are provided.

Further, the distal end portion 6 of the insertion portion 4 has a supporting portion 18 provided so as to project from the distal end face of the distal end portion 6, and the supporting portion 18 is positioned adjoining a lower part side of the cylindrical portion 10.

The supporting portion 18 is configured so as to be able to support or hold respective projecting members arranged so as to be projected from the distal end face of the distal end portion 6. More specifically, the supporting portion 18 is configured so as to be able to support or hold each of a forward observation window nozzle portion 19 configured to eject gas or liquid for cleaning the forward observation window 12 and lateral observation window nozzle portions 22 configured to eject gas or liquid for cleaning another forward illumination window 21 configured to emit light for illuminating the forward direction and the lateral observation window 13 as the respective projecting members described before.

On the other hand, the supporting portion 18 is formed having a shielding portion 18a which is an optical shielding member for preventing such a lateral field-of-view image that includes any of the respective projecting members described above from being acquired because the respective projecting members, which are objects different from the original observation target object, appear in the lateral field of view. That is, by providing the shielding portion 18a on the supporting portion 18, it is possible to obtain a lateral field-of-view image that does not include any of the forward observation window nozzle portion 19, the forward illumination window 21 and the lateral observation window nozzle portions 22.

As shown in FIGS. 2 and 3, the lateral observation window nozzle portions 22 are provided at two positions on the supporting portion 18 and arranged so that distal ends project from a side face of the supporting portion 18.

As shown in FIG. 1, the operation portion 3 is provided with an air/liquid feeding operation button 24a making it possible to give an operation instruction to eject gas or liquid for cleaning the forward observation window 12 from the forward observation window nozzle portion 19 and an air/liquid feeding operation button 24b making it possible to give an operation instruction to eject gas or liquid for cleaning the lateral observation window 13 from the lateral observation window nozzle portions 22, and it is possible to switch between air feeding and liquid feeding by pushing down the air/liquid feeding operation buttons 24a and 24b. Further, though a plurality of air/liquid feeding operation buttons are provided so as to correspond to the respective nozzle portions in the present embodiment, it is also possible to cause gas or liquid to be ejected from both of the forward observation window nozzle portion 19 and the lateral observation window nozzle portions 22, for example, by operating one air/liquid feeding operation button.

A plurality of scope switches 25 are provided on a top portion of the operation portion 3 and have a configuration in which, in order to cause signals corresponding to on, off and the like of various kinds of functions usable in the endoscope 2 to be outputted, the functions can be allocated to the respective switches. More specifically, for example, functions of causing signals corresponding to start and stop of forward water feeding, execution and release of freeze for photographing a still image, notification of a use state of a treatment instrument, and the like to be outputted can be allocated to the scope switches 25 as functions for the respective switches.

Note that, in the present embodiment, the function of at least one of the air/liquid feeding operation buttons 24a and 24b may be allocated to any of the scope switches 25.

Further, a suction operation button 26 making it possible to give an instruction to suck and collect mucus and the like in a body cavity from the distal end opening portion 17 to a suction unit or the like not shown is arranged on the operation portion 3.

Then, the mucus and the like in the body cavity sucked in response to an operation of the suction unit or the like not shown is collected to a suction bottle or the like of the suction unit not shown via the distal end opening portion 17, the treatment channel in the insertion portion 4, which is not shown, and a treatment instrument insertion port 27 provided near a front end of the operation portion 3.

The treatment instrument insertion port 27 communicates with the treatment instrument channel in the insertion portion 4, which is not shown, and formed as an opening through which a treatment instrument not shown can be inserted. That is, the operator can perform treatment using a treatment instrument by inserting the treatment instrument from the treatment instrument insertion port 27 and causing a distal end side of the treatment instrument to project from the distal end opening portion 17.

On the other hand, as shown in FIG. 1, a connector 29 connectable to the light source apparatus 31 is provided on the other end portion of the universal cord 5.

On a distal end portion of the connector 29, a pipe sleeve (not shown) to be a connection end portion of a fluid conduit and a light guide pipe sleeve (not shown) to be an illumination light supply end portion are provided. Further, on a side face of the connector 29, an electrical contact portion (not shown) to which one end portion of a connection cable 33 can be connected is provided. Furthermore, on the other end portion of the connection cable 33, a connector for electrically connecting the endoscope 2 and the video processor 32 is provided.

The universal cord 5 includes a plurality of signal lines for transmitting various kinds of electrical signals and a light guide for transmitting illumination light supplied from the light source apparatus 31 in a state of being bundled together.

The light guide included in the insertion portion 4 and the universal cord 5 has such a configuration that an end portion on a light emission side is branched in at least two directions near the insertion portion 4, and a light emission end face on one side is arranged at the forward illumination windows 16 and 21 and a light emission end face on the other side is arranged at the lateral direction illuminating portion 14. Further, the light guide has such a configuration that an end portion on a light incident side is arranged at the light guide pipe sleeve of the connector 29.

The video processor 32, which is an image processing apparatus and an image signal generation apparatus, outputs a drive signal for driving the image pickup device 40 provided on the distal end portion 6 of the endoscope 2. According to a use state of the endoscope 2, the video processor 32 generates a video signal by performing signal processing for an image pickup signal outputted from the image pickup device 40 (cutting out a predetermined area) and outputs the video signal to the monitor 35 as described later.

Peripheral apparatuses such as the light source apparatus 31, the video processor 32 and the monitor 35 are arranged on a stand 36 together with a keyboard 34 for performing input of patient information, and the like.

The light source apparatus 31 includes a lamp. Light emitted from the lamp is guided to a connector portion to which the connector 29 of the universal cord 5 is connected, via the light guide, and the light source apparatus 31 supplies illumination light to the light guide in the universal cord 5.

FIG. 4 shows an example of an endoscopic image displayed on the monitor 35. An observation image 35b, which is an endoscopic image displayed on a display screen 35a of the monitor 35, is a substantially rectangular image and has two parts 37 and 38. The circular part 37 in a central part is an area for displaying a forward field-of-view image, and the C-shaped part 38 around the 37 in the central part is a part for displaying a lateral field-of-view image.

Note that the image displayed in the part 37 and the image displayed in the part 38 in the endoscopic image displayed on the monitor 35 are not necessarily same as an image of an object in the forward field of view and an image of the object in the lateral field of view, respectively.

That is, the forward field-of-view image is displayed on the display screen 35a of the monitor 35 so as to be in a substantially circular shape, and the lateral field-of-view image is displayed on the display screen 35a so as to be in a substantially annular shape surrounding at least a part of a circumference of the forward field-of-view image. Therefore, a wide-angle endoscopic image is displayed on the monitor 35.

The endoscopic image shown in FIG. 4 is generated from an acquired image which has been acquired by the image pickup device 40 (FIG. 2). The observation image 35b is generated by photoelectrically converting an object image projected on the image pickup surface of the image pickup device 40 by the objective optical system provided in the distal end portion 6 and combining a part of an image in the forward field of view in the center, which corresponds to the part 37, and a part of an image in the lateral field of view, which corresponds to the part 38, excluding a mask area 39 which is painted black.

### (Configuration of Video Processor)

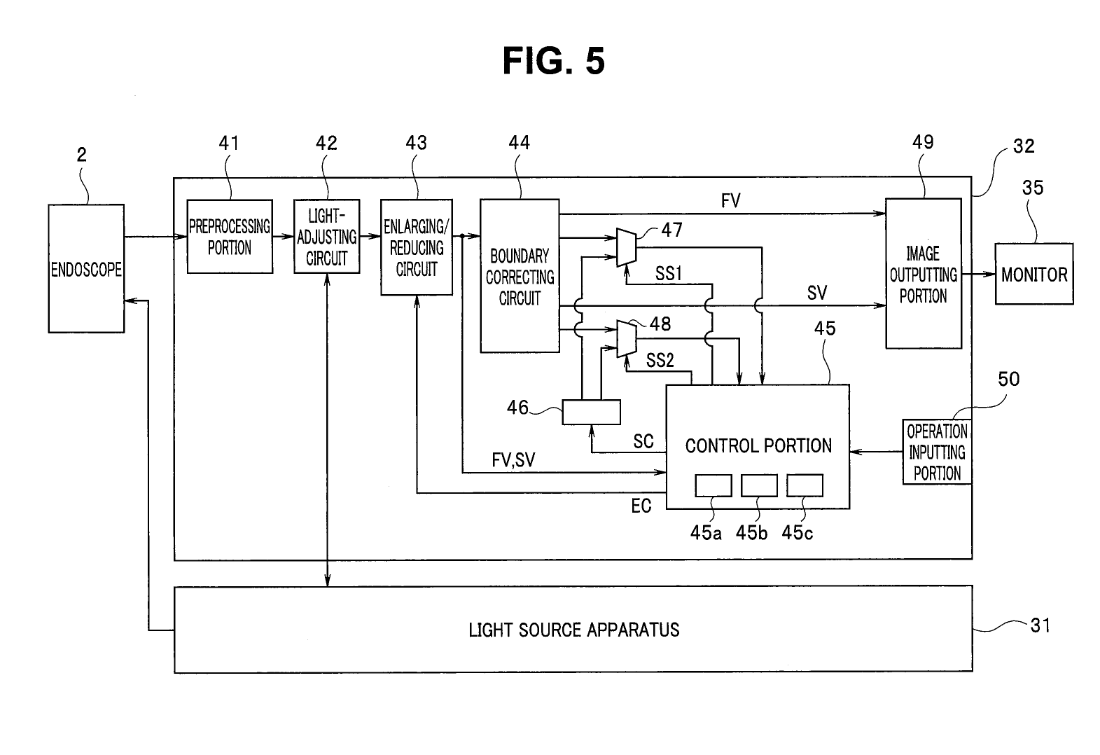

FIG. 5 is a block diagram showing a configuration of the video processor 32. In FIG. 5, only components that relate to functions of the present embodiment described below are shown, and components that relate to other functions such as image recording are omitted.

The video processor 32 has a preprocessing portion 41, a light-adjusting circuit 42, an enlarging/reducing circuit 43, a boundary correcting circuit 44, a control portion 45, a setting information storage portion 46, two selectors 47 and 48, an image outputting portion 49 and an operation inputting portion 50. As described later, the video processor 32 has a function of generating an image which has been image-processed.

The preprocessing portion 41 is a circuit configured to perform a process such as color filter conversion for an image pickup signal from the image pickup device 40 of the endoscope 2 and output a video signal for making it possible to perform various kinds of image processing in the video processor 32.

The light-adjusting circuit 42 is a circuit configured to judge brightness of an image based on the video signal and output a light adjustment control signal to the light source apparatus 31 based on a light adjustment state of the light source apparatus 31.

The enlarging/reducing circuit 43 cuts out a forward field-of-view image FV and a lateral field-of-view image SV from an image of the video signal outputted from the preprocessing portion 41 and supplies an image signal of the forward field-of-view image FV and an image signal of the lateral field-of-view image SV to the control portion 45. The enlarging/reducing circuit 43 enlarges or reduces the forward field-of-view image FV and the lateral field-of-view image SV according to a size and format of the monitor 35 and supplies the image signals of the forward field-of-view image FV and the lateral field-of-view image SV which have been enlarged or reduced to the boundary correcting circuit 44.

Furthermore, the enlarging/reducing circuit 43 is a circuit which is also capable of executing a process for enlarging or reducing areas set or specified in the forward field-of-view image FV and the lateral field-of-view image SV with a set or specified magnification based on a control signal EC from the control portion 45. Therefore, the control signal EC from the control portion 45 includes information about areas to be enlarged or reduced and enlargement or reduction magnification information.

The boundary correcting circuit 44 is a circuit configured to receive a video signal outputted from the enlarging/reducing circuit 43 and perform a necessary boundary correction process to separate and output the forward field-of-view image FV and the lateral field-of-view image SV. The image signal of the forward field-of-view image FV and the image signal of the lateral field-of-view image SV are supplied to the image outputting portion 49 and the control portion 45. Note that the boundary correcting circuit 44 also executes a masking process for defective pixels.

The boundary correction process executed by the boundary correcting circuit 44 is a process for, in a case of receiving the video signal outputted from the enlarging/reducing circuit 43 and outputting both of the forward field-of-view image FV and the lateral field-of-view image SV, performing a process for cutting out an image area in the forward field of view and an image area in the lateral field of view from the video signal based on boundary area information set in advance and an enlargement/reduction process for performing enlargement or reduction in order to correct a size of each cut-out image, for the video signal from the enlarging/reducing circuit 43 in order to display a boundary part between the forward field-of-view image FV and the lateral field-of-view image SV continuously and smoothly.

That is, in the case of outputting both of the forward field-of-view image FV and the lateral field-of-view image SV, the boundary correcting circuit 44 performs necessary boundary correction for the forward field-of-view image FV and the lateral field-of-view image SV which have been cut out and enlarged/reduced by the enlarging/reducing circuit 43 before outputting the forward field-of-view image FV and the lateral field-of-view image SV to the image outputting portion 49.

In a case of outputting only the forward field-of-view image FV, the boundary correcting circuit 44 does not execute the boundary correction process.

Further, the enlarging/reducing circuit 43 also holds default information about a pixel group or an area to be used by the control portion 45 to judge the use state of the endoscope 2 to be described later. That is, the enlarging/reducing circuit 43 can supply predetermined position information, that is, default information of a pixel group (hereinafter referred to as a judgment pixel group) or an area (hereinafter referred to as a judgment area) used to judge the use state of the endoscope 2 to the control portion 45 from each of the forward field-of-view image FV and the lateral field-of-view image SV via the boundary correcting circuit 44 and the selectors 47 and 48.

Note that the default information may be supplied to the control portion 45 from the enlarging/reducing circuit 43 not via the boundary correcting circuit 44 but only via the selectors 47 and 48.

The control portion 45 includes a central processing unit (CPU) 45a, a ROM 45b, a RAM 45c and the like. The control portion 45 executes a predetermined software program in response to a command or the like inputted to the operation inputting portion 50 by a user, generates or reads out various kinds of control signals and data signals and outputs the signals to appropriate circuits in the video processor 32.

Further, when the endoscope system 1 is set to an automatic image display switching mode to be described later by the user, the control portion 45 judges the use state of the endoscope 2 based on pixel values of a judgment pixel group or a judgment area set in one or both of the forward field-of-view image FV and the lateral field-of-view image SV outputted from the enlarging/reducing circuit 43 and, furthermore, generates and outputs a control signal SC which is a selection control signal to the setting information storage portion 46 and a control signal EC which is an enlargement/reduction control signal to the enlarging/reducing circuit 43 corresponding to the judged use state.

Here, the pixel values used for the judgment are values of pixels of at least one color among pixels of a plurality of colors of the image pickup device 40.

That is, the control portion 45 generates and outputs a control signal SC for selecting judgment pixel group information, judgment area information and the like to be used when the default information is not used, according to the judged use state.

The ROM 45b of the control portion 45 stores a display control program used during the automatic image display switching mode, and various kinds of information such as an evaluation formula for, in each use state, judging the use state is also written in the program or as data.

The control portion 45 stores information about a judged use state into a predetermined storage area in the RAM 45c.

The use state refers to a state of use of the endoscope 2 by the user, such as insertion of the insertion portion 4, screening for checking whether there is a lesion or not, suction of liquid and treatment of a living tissue by a treatment instrument.

An operation of the control portion 45 for judgment of the use state will be described later.

The setting information storage portion 46 is a memory or a register group configured to store a judgment pixel group or a judgment area set by the user (hereinafter referred to as user setting information) and user setting information about a mask area. The user can set the user setting information in the setting information storage portion 46 from the operation inputting portion 50.

The selector 47 is a circuit configured to select and output one of the default information from the enlarging/reducing circuit 43 and the user setting information set by the user, for the forward field-of-view image FV.

The selector 48 is a circuit configured to select and output one of the default information from the enlarging/reducing circuit 43 and the user setting information set by the user, for the lateral field-of-view image SV.

Whether the selectors 47 and 48 are to output the default information or the user setting information is decided by selection signals SS1 and SS2 from the control portion 45, respectively. The control portion 45 outputs the selection signals SS1 and SS2 so that the respective selectors 47 and 48 output the default information or the user setting information to be outputted, which is set according to a judged use state.

Respective pixels of a judgment pixel group set by the default information or the user setting information are pixels in an image area of the forward field-of-view image FV and an image area of the lateral field-of-view image SV, and pixels which cannot be used for judgment because of characteristics of the objective optical system are automatically removed or masked.

Furthermore, there may be a case where, in a certain use state, pixel values of pixels at a particular position are not used in order to judge change of the use state. For example, in order to make a judgment of a treatment instrument, change in pixel values only of a particular area in an image area, but, when the treatment instrument has been detected, pixel values of pixels of that area are excluded from pixels for judgment in order that the pixel values are not used for judgment of other use states. Therefore, according to a use state, the user can include pixels not to be used for judgment of other use states into the user setting information.

Further, a shape of the judgment area set by the user is not limited to a circle, a fan shape or a rectangle but may be any shape in each of the image area of the forward field-of-view image FV and the image area of the lateral field-of-view image SV.

A size and position of the judgment area set by the user may be arbitrary in each of the image area of the forward field-of-view image FV and the image area of the lateral field-of-view image SV. Pixels which cannot be used for judgment because of the characteristics of the objective optical system are automatically removed or masked. The user can set pixels or an area to be masked.

The boundary correcting circuit 44 outputs the forward field-of-view image FV and the lateral field-of-view image SV to the image outputting portion 49.

The image outputting portion 49 is a circuit as an image generating portion configured to combine the forward field-of-view image FV and the lateral field-of-view image SV from the boundary correcting circuit 44 to generate a composite image signal by image processing, convert the image signal to a display signal and output the display signal to the monitor 35.

Note that only the forward field-of-view image FV is outputted from the boundary correcting circuit 44, the image outputting portion 49 does not perform image combination.

The operation inputting portion 50 is an operation button, a keyboard and the like for the user to input various kinds of operation signals and various kinds of setting information. Information inputted to the operation inputting portion 50 is supplied to the control portion 45. For example, the user can input user setting information, settings for various kinds of automatic detection functions and the like to the control portion 45 using a keyboard to set them in the setting information storage portion 46.

Therefore, the user can perform setting of a judgment pixel group or a judgment area, setting of a mask area, setting of automatic detection of defective pixels, setting of automatic detection of foreign matter such as a treatment instrument and the like for each of use states of the endoscope 2 such as insertion and screening to be described later.

Furthermore, the user can also set information about whether the default information is to be used or the user setting information is to be used in each use state of the endoscope 2, in the setting information storage portion 46 as user setting information, and the control portion 45 controls output of the selection signals SS1 and SS2 based on the information.

Furthermore, the user can set a weighting factor to be described later in the setting information storage portion 46 as user setting information.

Further, the user can give instructions to execute various kinds of functions to the video processor 32 by performing predetermined input to the operation inputting portion 50, and can give an instruction to switch to the automatic image display switching mode to be described later to the video processor 32 by pressing down a predetermined operation button on the operation inputting portion 50.

Note that the operation inputting portion 50 may have a display portion such as a liquid crystal display.

The endoscope system 1 has a plurality of operation modes. When the endoscope system 1 is set to the automatic image display switching mode to be described later, the control portion 45 executes an observation image display control process corresponding to the automatic image display switching mode. When the endoscope system 1 is not set to the automatic image display switching mode, the control portion 45 executes an observation image display control process so that an observation image as shown in FIG. 4 is continuously displayed on the monitor 35.

The display when the endoscope system 1 is not set to the automatic image display switching mode is similar to that displayed conventionally. Therefore, here, description of the display is omitted, and the observation image display control process during the automatic image display switching mode will be described.

### (Operation)

Next, an operation of the endoscope system 1 during the automatic image display switching mode will be described.

An operation of the endoscope system 1 will be described below, with a case of inserting the insertion portion 4 into a large intestine to examine an inside of the large intestine as an example.

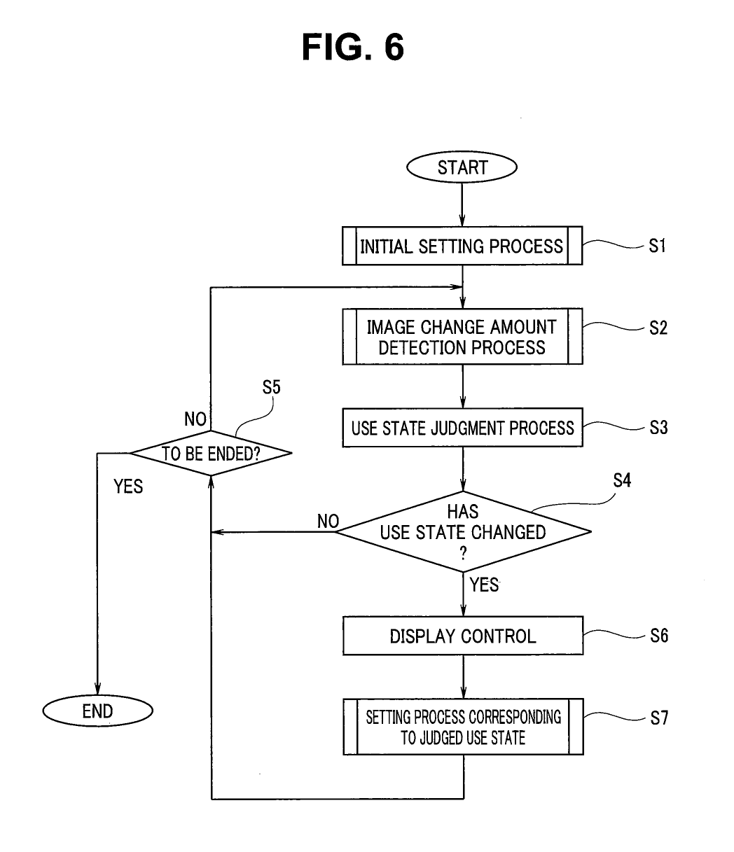

When the user sets the endoscope system 1 to the automatic image display switching mode on the operation inputting portion 50, a process of FIG. 6 is executed.

FIG. 6 is a flowchart showing an example of a flow of a whole process of the control portion 45 in the automatic image display switching mode in the endoscope system of the present embodiment.

The control portion 45 executes an initial setting process (S1). The initial setting process (S1) is a process for making it possible for the user to set a judgment area and a mask area. When the initial setting process (S1) is executed, for example, a predetermined menu screen for making it possible for the user to perform initial setting is displayed on the monitor 35.

In the initial setting process (S1), the user can select and set use of default information for a judgment area and a mask area. As for the mask area, the user can set whether or not to perform automatic detection of mask area.

When the user selects use of the default information, one or more judgment areas and one or more mask areas are automatically set.

When the default information is not to be used, the user can set one or more judgment areas for which image change in an initial state is to be detected and set one or more mask areas not to be used for judgment as a judgment area, for one or both of a forward field-of-view image, which is a first field-of-view image to be continuously displayed as a primary image, and a lateral field-of-view image, which is a second field-of-view image as a secondary image an display aspect of which is to be changed as necessary, for example, in accordance with an instruction of the menu screen displayed on the monitor 35.

The judgment area may be an arbitrary area in each of the forward field-of-view image and the lateral field-of-view image. For example, a whole image of each of the forward field-of-view image and the lateral field-of-view image, one of left and right sides, and the like can be set. The mask area is an area or pixels not to be used for detection of image change.

Note that, in the initial setting process (S1), an initial setting process for an initial parameter used in each circuit is also executed. For example, it is also possible to set parameters for the light-adjusting circuit 42, the enlarging/reducing circuit 43 and the like.

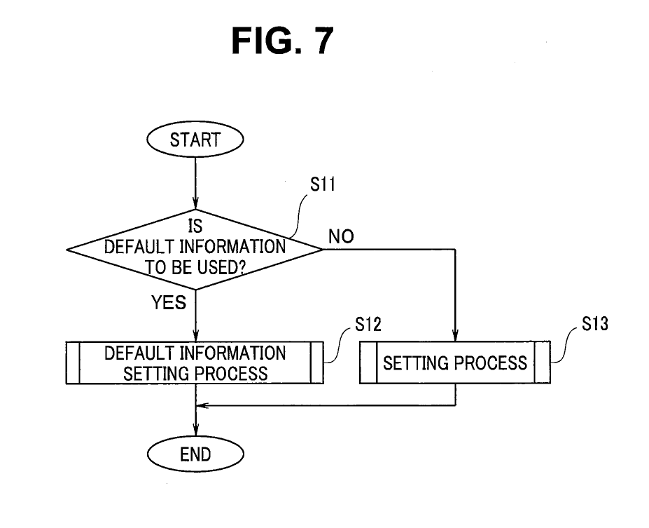

FIG. 7 is a flowchart showing an example of a flow of a judgment area setting process in the initial setting process (S1).

The control portion 45 judges whether the user has specified use of default information to set a judgment area, for example, on the predetermined menu screen for initial setting (S11). When use of the default information has been specified (S11: YES), the control portion 45 executes a default information setting process for causing a judgment area as default information set in the ROM 45b or each circuit to be used in the initial state (S12).

When use of the default information is not specified (S11: NO), the control portion 45 executes a setting process for making it possible for the user to set a judgment area, by displaying a menu screen or the like for the user to set a judgment area on the monitor 35 or on the operation inputting portion 50 (S13).

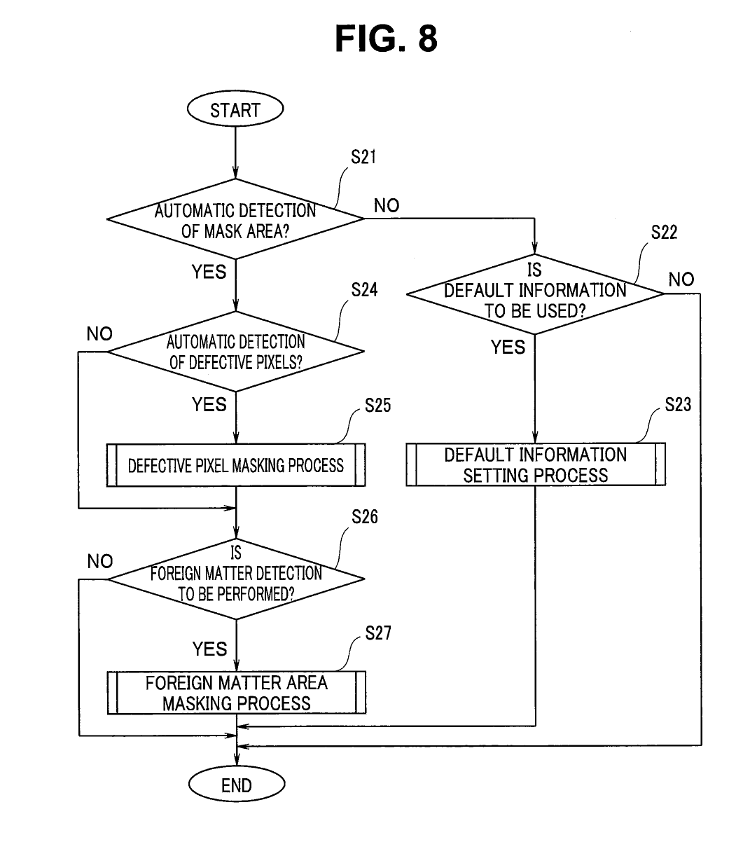

FIG. 8 is a flowchart showing an example of a flow of a mask area setting process in the initial setting process (S1). The process of FIG. 8 is executed by the user inputting settings on a menu screen.

The control portion 45 judges whether automatic detection of mask area has been specified (S21). The control portion 45 can judge whether the user has specified automatic detection of mask area or not, for example, on the predetermined menu screen for initial setting.

When automatic detection of mask area is not specified (S21: NO), the control portion 45 judges whether use of default information is specified (S22). The control portion 45 can judge whether or not the user has specified use of default information for a mask area, for example, on the predetermined menu screen for initial setting.

When use of default information is specified (S22: YES), the control portion 45 executes a default information setting process for causing a mask area as default information set in the ROM 45b or each circuit to be used in the initial state (S23).

When use of the default information is not specified (S22: NO), the control portion 45 does not perform any process, and a mask area is not set as a result.

When automatic detection of mask area is specified (S21: YES), the control portion 45 judges whether automatic detection of defective pixels is set (S24). The control portion 45 can make the judgment based on whether or not the user has specified automatic detection of mask area, for example, on the predetermined menu screen for initial setting.

When automatic detection of defective pixels is set (S24: YES), the control portion 45 performs detection of defective pixels and executes a defective pixel masking process for performing a masking process for the defective pixels (S25).

When automatic detection of defective pixels is not specified (S24: NO), the control portion 45 judges whether it is specified to perform a foreign matter detection process (S26). A foreign matter is, for example, a treatment instrument. The control portion 45 can make the judgment based on whether the user has specified detection of foreign matter, for example, on the predetermined menu screen for initial setting.

When it is specified to perform the foreign matter detection process (S26: YES), the control portion 45 executes a foreign matter area masking process for performing a masking process for a predetermined area for detecting a foreign matter (S27).

Returning to FIG. 6, the control portion 45 executes an image change amount detection process (S2) after the initial setting (S1). After the initial setting (S1), an observation image as shown in FIG. 4 is displayed on the monitor 35. After the initial setting ends, no use state is set yet. Then, the image change amount detection process is executed (S2) based on the setting information set by the initial setting process (S1).

After the initial setting (S1), the user inserts the distal end portion 6 of the insertion portion 4 from an anus and inserts the distal end portion 6 to a deepest part of an examination target area inside a large intestine.

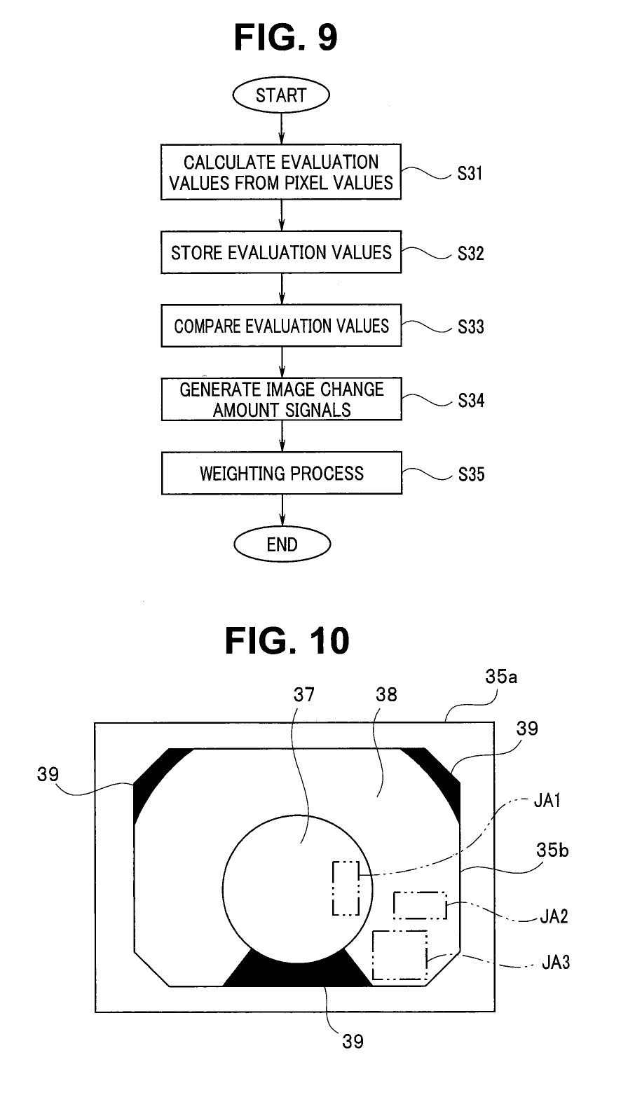

FIG. 9 is a flowchart showing an example of a flow of the image change amount detection process (S2). After the initial setting (S1), the control portion 45 executes the process of FIG. 9 based on the setting information set by the initial setting (S1). Here, description will be made about a case where a judgment area is set.

For each inputted frame, the control portion 45 calculates a predetermined evaluation value from pixel values of a pixel group in the judgment area (S31). At this time, pixel values of pixels of a mask area are not used for calculation of the evaluation value.

A method for calculating the evaluation value is set in advance for each judgment area.

The control portion 45 stores each calculated evaluation value into a predetermined storage area (S32). The predetermined storage area is the RAM 45c, a frame buffer not shown or the like in the control portion 45. Each evaluation value is stored in the predetermined storage area in association with frames of the forward field-of-view image and the lateral field-of-view image for which the evaluation value has been calculated.

The control portion 45 compares an evaluation value of each judgment area of a current frame and an evaluation value of each judgment area of a frame immediately before the current frame (S33).

The control portion 45 generates image change amount signals from a result of the comparison at S33 (S34).

The control portion 45 performs a weighting process for each of the generated two image change amount signals (S35). Here, since a weighting factor is used, use state judgment can be performed more appropriately according to a use state.

Here, an example of a process of S31 to S35 will be described.

FIG. 10 is a diagram for illustrating a set judgment area in an endoscopic image displayed on the monitor 35. In FIG. 10, a judgment area JA1 indicated by a two-dot chain line is set in the forward field-of-view image part 37, and two judgment areas JA2 and JA3 indicated by two-dot chain lines are set in the lateral field-of-view image part 38. The judgment area JA3 is an area which is also used to detect projection of a distal end portion of a treatment instrument, which is a foreign matter from the distal end opening portion 17 of the distal end portion 6 of the insertion portion 4.

The evaluation value calculated at S31 is calculated, for example, for each of the judgment areas JA1, JA2 and JA3 of each frame. Respective evaluation values s1 and s2 of the judgment areas JA1 and JA2 are sum totals of pixel values of pixel groups included in the judgment areas JA1 and JA2, respectively. An evaluation value s3 of the judgment areas JA3 indicates a magnitude of an edge component calculated from pixel values of a pixel group included in the judgment area JA3. The evaluation values s1, s2 and s3 of the respective judgment areas JA1, JA2 and JA3 are stored for each frame.

Note that, though the sum total of the pixel values of the pixel group included in each of the judgment areas JA1 and JA2 is used as the evaluation value here, an average value of the pixel values of the pixel group included in each of the judgment areas JA1 and JA2 may be used as the evaluation value.

For example, the comparison performed at S33 is, for each frame, calculation of differences ds1 and ds2 between the evaluation values s1 and s2 of the current frame and the evaluation values s1 and s2 of an immediately preceding frame for the respective judgment areas JA1 and JA2.

As for the judgment area JA3, the comparison is calculation of a difference ds3 between magnitudes of an edge component of the current frame and an edge component of the immediately preceding frame.

The image change amount signals generated at S34 are signals d1, d2 and d3 indicating the calculated differences ds1, ds2 and ds3 for the respective judgment areas JA1, JA2 and JA3.

In the weighting process performed at S35, the image change amount signal dl which is a difference calculated for the judgment area JA1, the image change amount signal d2 which is a difference calculated for the judgment area JA2 and the image change amount signal d3 which is a difference calculated for the judgment area JA3 are multiplied by predetermined weighting factors c1, c2 and c3, respectively, and weighted image change amount signals wd1, wd2 and wd3 are calculated and obtained.

That is, for the respective judgment areas JA1, JA2 and JA3 shown in FIG. 10, the weighted image change amount signals wd1, wd2 and wd3 are calculated and obtained for each frame from comparison with evaluation values of an immediately preceding frame. Therefore, a use state of the insertion portion 4 is judged based on a detection result obtained by weighting the detected image change amount signals d1, d2 and d3.

While the user inserts the distal end portion 6 of the insertion portion 4 from the anus and inserts the distal end portion 6 to the deepest part of the examination target area in the large intestine after the initial setting, change in the forward field-of-view image and the lateral field-of-view image is large.

Therefore, evaluation values largely change among frames, and values of the weighted image change amount signals wd1 and wd2 also increase. The image change amount signal wd3 does not change largely. Note that magnitudes of weighting of the image change amount signals d1, d2 and d3 are same here.

As described above, the process of S2 constitutes an image change amount detecting portion configured to detect amounts of change of pixel values, which are color information about image signals in the predetermined judgment areas JA1, JA2 and JA3 in at least one of the forward field-of-view image and the lateral field-of-view image within a predetermined time period.

Returning to FIG. 6, the control portion 45 executes a use state judgment process based on the image change amount signals calculated in the image change amount detection process shown in FIG. 9 after S2 (S3).

For example, while the insertion portion 4 is being inserted deep in the large intestine, the distal end portion 6 advances in the large intestine while operations of advancing and withdrawing the insertion portion 4 is repeated by the user. Therefore, the evaluation values s1 and s2 of the judgment areas JA1 and JA2 of each of the obtained forward field-of-view image and lateral field-of-view image continue changing largely.

Therefore, when change in the image change amount signals wd1 and wd2 equal to or above a predetermined threshold TH1 continues within a predetermined time period T1 as described above, the control portion 45 judges that the use state is an insertion state.

As described above, the control portion 45 makes a judgment of the use state of the endoscope 2 based on the image change amount signals wd1, wd2 and wd3. Therefore, the process of S3 constitutes a use state judging portion configured to judge the use state of the insertion portion 4 based on a detection result of the image change amount detecting portion.

After S3, the control portion 45 judges whether the use state has changed or not (S4).

When the change as described above is not detected, the control portion 45 assumes that there is not a change in the use state (S4: NO) and judges whether an instruction to end the automatic image display switching mode has been given or not (S5). The instruction to end the automatic image display switching mode is given by the user on the operation inputting portion 50.

When the instruction to end the automatic image display switching mode is not given (S5: NO), the process proceeds to S2.

Further, when it is judged that the use state has changed (S4: YES), the control portion 45 executes display control (S6). The control portion 45 generates and outputs a control signal EC to the enlarging/reducing circuit 43 so as to display an observation image in a display format set in advance on the monitor 35, according to a judged use state. For example, in the case of the insertion state, the control portion 45 generates a control signal EC for displaying only the forward field-of-view image which has been enlarged, on the monitor 35 and outputs the control signal EC to the enlarging/reducing circuit 43.

After S6, the control portion 45 executes a setting process for setting a control signal SC in the setting information storage portion 46 in order to select and output a judgment area and the like set in advance in the setting information storage portion 46, according to the judged use state (S7). After S7, the process proceeds to S5.

Next, use states to be judged will be specifically described.

### (Insertion State)

After the initial state, when change in the image change amount signals wd1 and wd2 within the predetermined time period T1 as described above is equal to or above the predetermined threshold TH1, the control portion 45 judges that the use state has changed (S4: YES), and after storing insertion state information into a predetermined storage area on the RAM 45c as use state information, executes display control corresponding to the judged use state (S6).

For example, when it is judged that the user is performing an operation of inserting the endoscope 2, the control portion 45 executes display control so that only the forward field-of-view image which has been enlarged is displayed on the monitor 35. When the user is inserting the insertion portion 4 into the large intestine, which is a lumen, an image which the user is mainly interested in is the forward field-of-view image. Therefore, the user can perform the insertion operation more quickly and more certainly by causing only the forward field-of-view image to be enlarged and displayed on the monitor 35 without causing the lateral field-of-view image to be displayed during insertion.

Therefore, the control portion 45 outputs a magnification for causing the forward field-of-view image to be displayed large on the monitor 35 and a control signal EC for preventing the lateral field-of-view image from being displayed to the enlarging/reducing circuit 43.

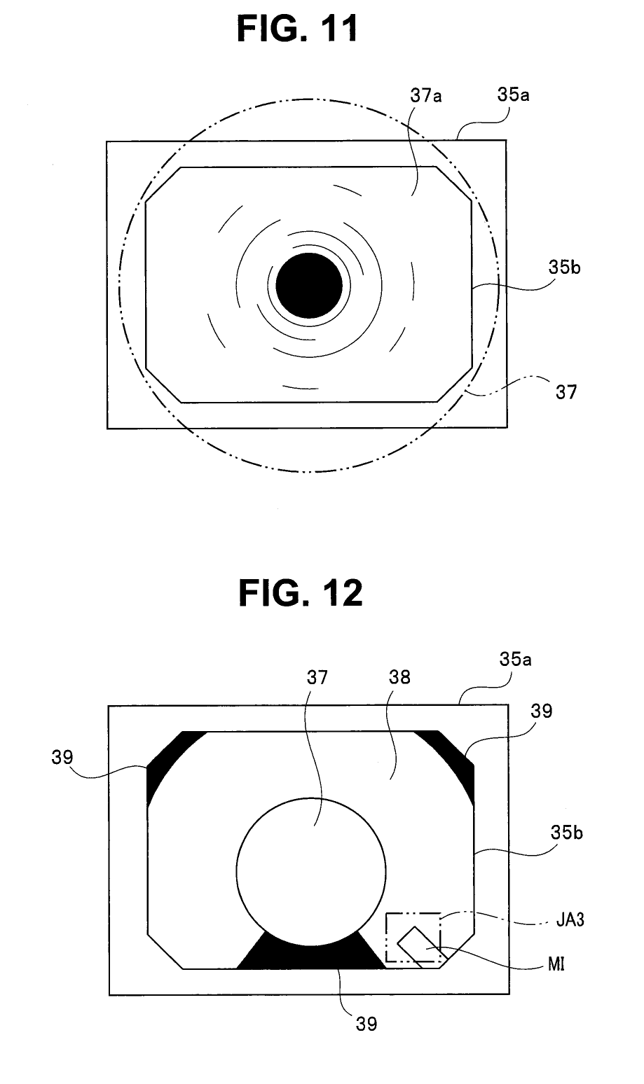

FIG. 11 is a diagram showing an example of an endoscopic image displayed on the monitor 35 in the insertion state. The forward field-of-view image part 37 indicated by a two-dot chain line is enlarged; the lateral field-of-view image part 38 is hidden; and a part 37a in a center of the forward field-of-view image part 37 is displayed as an observation image 35b on the display screen 35a of the monitor 35. In a center part of the observation image 35b, an area inside a lumen tip is displayed as a dark part.

When display control is executed, it is judged whether an instruction to end the automatic image display switching mode has been given or not (S5). When the instruction to end the automatic image display switching mode has not been given, the process proceeds to S2.

### (Screening State)

When the distal end portion 6 of the insertion portion 4 reaches the deepest part of the large intestine, the insertion portion 4 is slowly pulled out, and, therefore, change in the evaluation values of the judgment areas JA1 and JA2 of each of the forward field-of-view image and the lateral field-of-view image obtained in the image change amount detection process (S2) becomes small.

Therefore, when such a large change that the change in the image change amount signals wd1 and wd2 within the predetermined time period T1 is equal to or above the predetermined threshold TH1 has ceased, and such a small change that the change in the image change amount signals wd1 and wd2 within the predetermined time period T1 is below the predetermined threshold TH1 and equal to or above a predetermined threshold TH2 continues, the control portion 45 judges that the use state is a screening state (S3).

Since the use state has changed from the insertion state to the screening state (S4: YES), the control portion 45 executes display control according to the judged use state (S6).

At time of screening, the user has to certainly see not only the forward field-of-view image but also the lateral field-of-view image to confirm presence or absence of a lesion and the like. Therefore, the control portion 45 executes display control so that the forward field-of-view image and the lateral field-of-view image as shown in FIG. 4 are displayed on the monitor 35.

After the process of S6, the process proceeds to S2 if the instruction to end the automatic image display switching mode is not given.

### (Treatment Instrument Used State)

After execution of S6, the control portion 45 executes use state judgment again based on the inputted image change amount signals wd1, wd2 and wd3 at S2.

There may be a case where the user performs treatment using a treatment instrument. A distal end portion of the treatment instrument projects from the distal end opening portion 17 of the distal end portion 6. Therefore, it is judged whether or not the use state is a state of treatment using a treatment instrument, based on whether the image change amount signal wd3 in a predetermined area in the lateral field-of-view image which includes the judgment area JA3 has shown a predetermined change or not.

Since a treatment instrument is generally made of metal such as stainless steel, luminance of an image area of the treatment instrument is high in an endoscopic image. Therefore, the control portion 45 judges presence or absence of an edge area in the judgment area JA3 and, when strengths (slopes) of edges in an immediately preceding frame and a current frame exceed a threshold TH3, judges that the distal end portion of the treatment instrument has projected from the distal end opening portion 17.

FIG. 12 is a diagram showing an example of an observation image when a treatment instrument appears in an observation image when screening is performed. FIG. 12 shows a state in which a treatment instrument MI projects from a predetermined position of the lateral field-of-view image part 38 in the observation image and is positioned in the judgment area JA3.

Since the treatment instrument MI is displayed bright in the observation image, an edge area is detected in the judgment area JA3.

Therefore, when a difference between a size or length of the edge area in a current frame and a size or length of the edge area in an immediately preceding frame exceeds the predetermined threshold TH3, the control portion 45 judges that a distal end portion of the treatment instrument MI has projected from the distal end opening portion 17 and can judge at S3 that the use state is a treatment instrument used state.

When it is judged that the use state is the treatment instrument used state, the control portion 45 judges that the use state has changed (S4: YES). After storing treatment instrument used state information into a predetermined storage area on the RAM 45c as use state information, the control portion 45 executes display control corresponding to the judged use state (S6).

For example, since the user is using the treatment instrument MI, the control portion 45 executes display control so that only the lateral field-of-view image in which an area around the treatment instrument MI is enlarged is displayed on the monitor 35 (S6).

The control portion 45 outputs a magnification for causing a predetermined area in the lateral field-of-view image which includes the treatment instrument MI to be displayed large on the monitor 35 and a control signal EC for reducing a range in which the forward field-of-view image is displayed to the enlarging/reducing circuit 43.

As a result, since an image of the treatment instrument MI which the user is mainly interested in is displayed large, the user can perform treatment by the treatment instrument MI quickly and certainly.

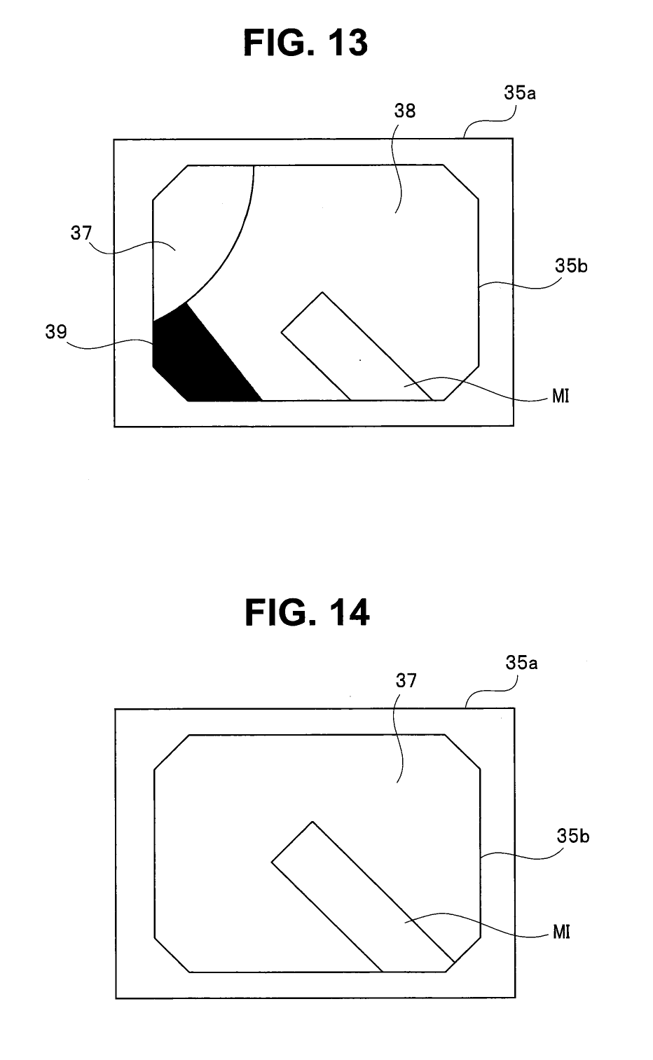

FIG. 13 is a diagram showing an example of an observation image displayed on the monitor 35 in the treatment instrument used state. An observation image 35b is displayed which is an endoscopic image where a partial area of the lateral field-of-view image part 38 which includes the treatment instrument MI is enlarged.

Note that it is also possible to set a judgment area for detecting that the distal end portion of the treatment instrument MI has entered the forward field-of-view image part 37, in the forward field-of-view image part 37 and perform display control so that only the forward field-of-view image part 37 which includes the treatment instrument MI is enlargedly displayed.

FIG. 14 is a diagram showing an example of an observation image displayed on the monitor 35 in the treatment instrument used state. An observation image 35b is displayed which is an endoscopic image where a partial area of the forward field-of-view image part 37 which includes the treatment instrument MI is enlarged, and the lateral field-of-view image part 38 is hidden.

Note that when the treatment instrument MI is withdrawn into the distal end opening portion 17 of the distal end portion 6, the control portion 45 does not detect the treatment instrument MI any longer, and display control is performed so that the observation image on the monitor 35 returns to such as is shown, for example, in FIG. 4.

### (Suction State)

There may be a case where suction of liquid is performed during endoscopy. For example, the operator may want to suck liquid such as cleaning liquid in a lumen to clean an inside of the lumen. When there is liquid in an endoscopic image, liquid-specific image change occurs in an image area where the liquid exists.

It is possible to set a judgment area for liquid judgment in the forward field-of-view image in advance and, on an assumption that change in pixel values in the judgment area is change indicating existence of liquid, judge existence of liquid, for example, based on presence or absence of predetermined luminance change within a predetermined time period.

Note that it is also possible to, instead of judging presence or absence of liquid only by image processing, make a judgment of the suction state when two conditions are satisfied at S3 that liquid has been detected by image processing, and that the suction operation button 26 has been operated.

As described above, the process of S6 constitutes an image control portion configured to, according to a judged use state, control the image signal of the forward field-of-view image and the image signal of the lateral field-of-view image outputted to the monitor 35 which is a display portion capable of displaying the forward field-of-view image and the lateral field-of-view image to perform display, non-display, partial enlargement and the like.

Therefore, at S2, the amounts of change in the image signals in the predetermined judgment areas JA1, JA2 and JA3 in at least one of the forward field-of-view image and the lateral field-of-view image displayed on the monitor 35 within the predetermined time period T1 are detected.

Furthermore, at S2, image change in the lateral field-of-view image which is not displayed on the monitor 35 is also detected, for example, in the insertion state in order to detect change to another use state. At S2, the amounts of change in the image signals in the predetermined judgment areas JA1, JA2 and JA3 in the forward field-of-view image or the lateral field-of-view image which is not displayed on the monitor 35 within the predetermined time period T1 can be also detected.

That is, in order to, even if a state of not displaying a secondary image continues, cause the secondary image to be displayed again when the use state of the endoscope 2 changes, the video processor 32 continues detecting amounts of change in a primary image and the secondary image though the secondary image is not caused to be displayed.

Here, the use state to be judged is any of a state in which insertion of the insertion portion 4 into an inside of an object is being performed, a state in which the distal end portion 6 of the insertion portion 4 is slowly moving in the inside of the object, a state in which a treatment instrument is projected from the distal end portion 6 of the insertion portion 4 and a state in which liquid inside the object is sucked from the distal end portion 6 of the insertion portion 4.

As described above, according to the embodiment described above, it is possible to provide an endoscope system capable of displaying an observation image an information amount of which has been controlled to be an optimum information amount required, according to a use state of an endoscope.

Note that, though a lateral field-of-view image is acquired with use of a double-reflection optical system as a method for forming a forward field-of-view image and the lateral field-of-view image on one image pickup device in the present embodiment, a single-reflection optical system may be used to acquire the lateral field-of-view image. In the case of using the single-reflection optical system, a direction of the lateral field-of-view image may be adjusted by image processing or the like as necessary.

### Second Embodiment

Though the endoscope system 1 of the first embodiment uses the endoscope 2 which obtains a forward field-of-view image and a lateral field-of-view image arranged surrounding the forward field-of-view image with one image pickup device, an endoscope system 1A of a second embodiment uses an endoscope 2A which obtains a forward field-of-view image and a lateral field-of-view image with separate image pickup devices.

Note that, in a configuration of the endoscope system 1A of the second embodiment, same components as those of the endoscope system 1 described in the first embodiment will be given same reference numerals, and description of the components will be omitted.

### (Configuration)

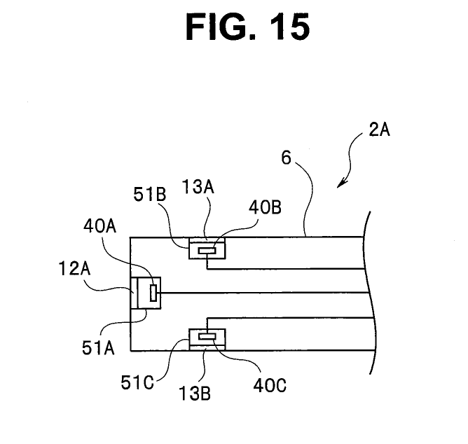

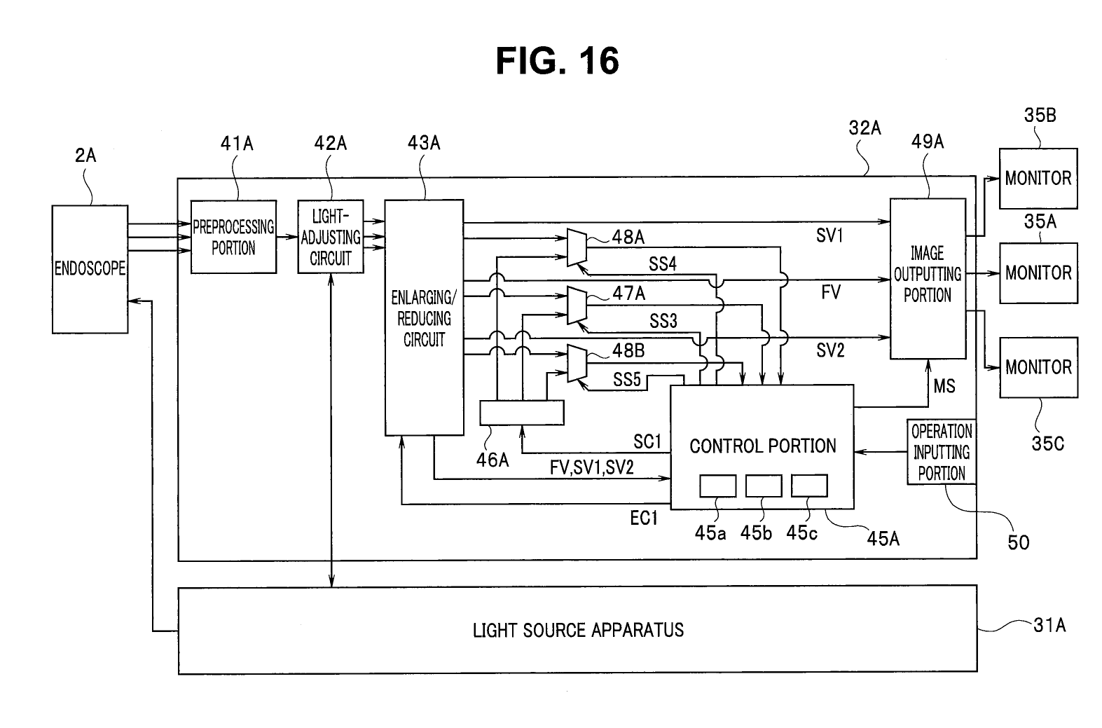

FIG. 15 is a schematic diagram showing a configuration of a distal end portion 6 of the endoscope 2A of the present embodiment. FIG. 16 is a block diagram showing a configuration of a video processor 32A according to the present embodiment. Note that, in FIG. 16, only components that relate to functions of the present embodiment described below are shown, and components that relate to other functions such as image recording are omitted.

As shown in FIG. 16, the endoscope system 1A includes the endoscope 2A, the video processor 32A, a light source apparatus 31A and three monitors 35A, 35B and 35C.

A configuration of the distal end portion 6 of the endoscope 2A will be described first. As shown in FIG. 15, a distal end face of the columnar distal end portion 6 of the endoscope 2A is provided with an image pickup unit 51A for forward field of view. A side face of the distal end portion 6 of the endoscope 2A is provided with two image pickup units 51B and 51C for lateral field of view. The three image pickup units 51A, 51B and 51C have image pickup devices 40A, 40B and 40C, respectively, and each image pickup unit is provided with an objective optical system not shown.

The image pickup units 51A, 51B and 51C are arranged on back sides of a forward observation window 12A and lateral observation windows 13A and 13B, respectively. The respective image pickup units 51A, 51B and 51C receive reflected light from an object illuminated by illumination light emitted from three illumination windows not shown and output image pickup signals.

The three image pickup signals from the three image pickup devices 40A, 40B and 40C are inputted to a preprocessing portion 41A.

The forward observation window 12A is arranged on the distal end portion 6 of the insertion portion 4, facing a direction in which the insertion portion 4 is inserted. The lateral observation windows 13A and 13B are arranged on a side face portion of the insertion portion 4 facing an outer diameter direction of the insertion portion 4 and at substantially equal angles in a circumferential direction of the distal end portion 6, and the lateral observation windows 13A and 13B are arranged so as to face mutually opposite directions on the distal end portion 6.

The image pickup devices 40A, 40B and 40C of the image pickup units 51A, 51B and 51C are electrically connected to the video processor 32A and controlled by the video processor 32A to output image pickup signals to the video processor 32A. Each of the image pickup units 51A, 51B and 51C is an image pickup portion configured to photoelectrically convert an object image.

The forward observation window 12A is provided on the distal end portion 6 in a longitudinal direction of the insertion portion 4 and constitutes a first image acquiring portion configured to acquire a first object image from a first area which includes the direction in which the insertion portion 4 is inserted (a forward direction), which is a first direction. In other words, the forward observation window 12A is a forward image acquiring portion configured to acquire an object image of an area which includes a forward direction of the insertion portion 4, and the first object image is an object image of an area which includes the forward direction of the insertion portion 4 almost parallel to the longitudinal direction of the insertion portion 4.

Each of the lateral observation windows 13A and 13B is provided on the distal end portion 6 in the longitudinal direction of the insertion portion 4 and constitutes a second image acquiring portion configured to acquire a second object image from a second area which includes a lateral direction of the insertion portion 4 which is a second direction different from the first direction. In other words, each of the lateral observation windows 13A and 13B is a lateral image acquiring portion configured to acquire an object image of an area which includes a direction crossing the longitudinal direction of the insertion portion 4, for example, at right angles, and the second object image is an object image of an area which includes the lateral direction of the insertion portion 4 which is a direction crossing the longitudinal direction of the insertion portion 4.

The image pickup unit 51A is an image pickup portion configured to photoelectrically convert an image from the forward observation window 12A, and the image pickup units 51B and 51C are image pickup portions configured to photoelectrically convert two images from the lateral observation windows 13A and 13B, respectively. That is, the image pickup unit 51A is an image pickup portion configured to pick up an object image for acquiring a forward field-of-view image, and each of the image pickup units 51B and 51C is an image pickup portion configured to pick up an object image for acquiring a lateral field-of-view image. An image signal of the forward field-of-view image, which is a first field-of-view image to be continuously displayed as a primary image is generated from an image obtained by the image pickup unit 51A, and image signals of the two lateral field-of-view images, which are second field-of-view images as secondary images the display aspect of which are to be changed as necessary, are generated from images obtained by the image pickup units 51B and 51C.

On a back side of each illumination window (not shown), a light-emitting device for illumination is arranged in the distal end portion 6 though it is not shown. The light-emitting device for illumination (hereinafter referred to as the light-emitting device) is, for example, a light emitting diode (LED). Therefore, the light source apparatus 31A has a driving portion configured to drive each light-emitting device.

As shown in FIG. 16, the video processor 32A has the preprocessing portion 41A, a light-adjusting circuit 42A, an enlarging/reducing circuit 43A, a control portion 45A, a setting information storage portion 46A, three selectors 47A, 48A and 48C, an image outputting portion 49A and an operation inputting portion 50. The video processor 32A has a function of generating an image which has been image-processed.

The preprocessing portion 41A is a circuit configured to perform a process such as color filter conversion for an image pickup signal from each of the image pickup devices 40A, 40B and 40C of the endoscope 2A and output video signals for making it possible to perform various kinds of processing in the video processor 32A.

The light-adjusting circuit 42A is a circuit configured to judge brightness of images based on the respective video signals of three object images and output a light adjustment control signal to the light source apparatus 31A based on a light adjustment state of the light source apparatus 31A.

The enlarging/reducing circuit 43A supplies image signals of a forward field-of-view image FV and two lateral field-of-view images SV1 and SV2 of the respective video signals outputted from the preprocessing portion 41A to the control portion 45A, and enlarges or reduces the forward field-of-view image FV and the two lateral field-of-view images SV1 and SV2 according to respective sizes and formats of the monitors 35A, 35B and 35C to supply an image signal of the enlarged or reduced forward field-of-view image FV and image signals of the two enlarged or reduced lateral field-of-view image SV1 and SV2 to the image outputting portion 49A.

Furthermore, the enlarging/reducing circuit 43A is a circuit which is also capable of executing a process for enlarging or reducing areas set or specified in each image with a set or specified magnification based on a control signal EC1 which is an enlargement/reduction control signal from the control portion 45A. Therefore, the control signal EC1 from the control portion 45A includes information about areas to be enlarged or reduced and enlargement or reduction magnification information about each image.

Further, the enlarging/reducing circuit 43A also holds default information of a judgment pixel group or a judgment area to be used by the control portion 45A to judge a use state of the endoscope 2A at the control portion 45A. That is, the enlarging/reducing circuit 43A can supply predetermined position information, that is, default information of a pixel group or an area used to judge the use state of the endoscope 2A to the control portion 45 from each of the forward field-of-view image FV and the two lateral field-of-view images SV1 and SV2 via the selectors 47A, 48A and 48B.

Similarly to the control portion 45 of the first embodiment, the control portion 45A includes a central processing unit (CPU) 45a, a ROM 45b, a RAM 45c and the like. The control portion 45A executes a predetermined software program in response to a command or the like inputted to the operation inputting portion 50 by a user, generates or reads various kinds of control signals and data signals and outputs the signals to appropriate circuits in the video processor 32A.

Further, when the endoscope system 1A is set to an automatic image display switching mode by the user, the control portion 45A judges the use state of the endoscope 2A based on pixel values of a judgment pixel group or a judgment area set in one or more of the forward field-of-view image FV and the lateral field-of-view images SV1 and SV2 outputted from the enlarging/reducing circuit 43A and, furthermore, generates and outputs a control signal SC1 to the set information storage portion 46A and a control signal EC1 to the enlarging/reducing circuit 43A corresponding to the judged use state.

That is, the control portion 45A generates and outputs a control signal SC1 for selecting judgment pixel group information, judgment area information and the like to be used when the default information is not used, according to the judged use state.

The ROM 45b of the control portion 45A stores a display control program used during the automatic image display switching mode, and various kinds of information such as an evaluation formula for, in each use state, judging the use state is written in the program or as data.

The control portion 45A stores information about a judged use state into a predetermined storage area in the RAM 45c.

Similarly to the setting information storage portion 46 of the first embodiment, the setting information storage portion 46A is a memory or a register group configured to store user setting information set by the user and user setting information about a mask area. The user can set the user setting information in the set information storage portion 46A from the operation inputting portion 50.

The selector 47A is a circuit configured to select and output one of the default information from the enlarging/reducing circuit 43A and the user setting information set by the user, for the forward field-of-view image FV.

The selectors 48A and 48B are circuits configured to select and output one of the default information from the enlarging/reducing circuit 43A and the user setting information set by the user, for the lateral field-of-view images SV1 and SV2, respectively.

Whether the selectors 47A, 48A and 48B are to output the default information or the user setting information is decided by selection signals SS3, SS4 and SS5 from the control portion 45A, respectively. The control portion 45A outputs the selection signals SS3, SS4 and SS5 so that the respective selectors 47A, 48A and 48B output the default information or the user setting information to be outputted, which is set according to a judged use state.

Respective pixels of a judgment pixel group set by the default information or the user setting information are pixels in an image area of the forward field-of-view image FV and image areas of the lateral field-of-view images SV1 and SV2, and pixels which cannot be used for judgment because of characteristics of the objective optical system are automatically removed or masked.

A size and position of the judgment area set by the user can be set in each image area, and a shape of the set judgment area is not limited to a circle and a rectangle but may be any shape in each of the image area of the forward field-of-view image FV and the image areas of the two lateral field-of-view images SV1 and SV2.

The enlarging/reducing circuit 43A outputs the forward field-of-view image FV, the lateral field-of-view image SV1 on a right side and the lateral field-of-view image SV2 on a left side not only to the image outputting portion 49A but also to the control portion 45A.

The image outputting portion 49A is a circuit configured to generate video signals of the forward field-of-view image FV and the two lateral field-of-view image SV1 and SV2 from the enlarging/reducing circuit 43A and outputs the video signals to the three monitors 35A, 35B and 35C based on a monitor selection signal MS which is a control signal from the control portion 45A.

The forward field-of-view image FV and the two lateral field-of-view image SV1 and SV2 generated by the video processor 32A are displayed on the monitors 35A, 35B and 35C. Therefore, wide-angle endoscopic images are displayed on the monitors 35A, 35B and 35C.

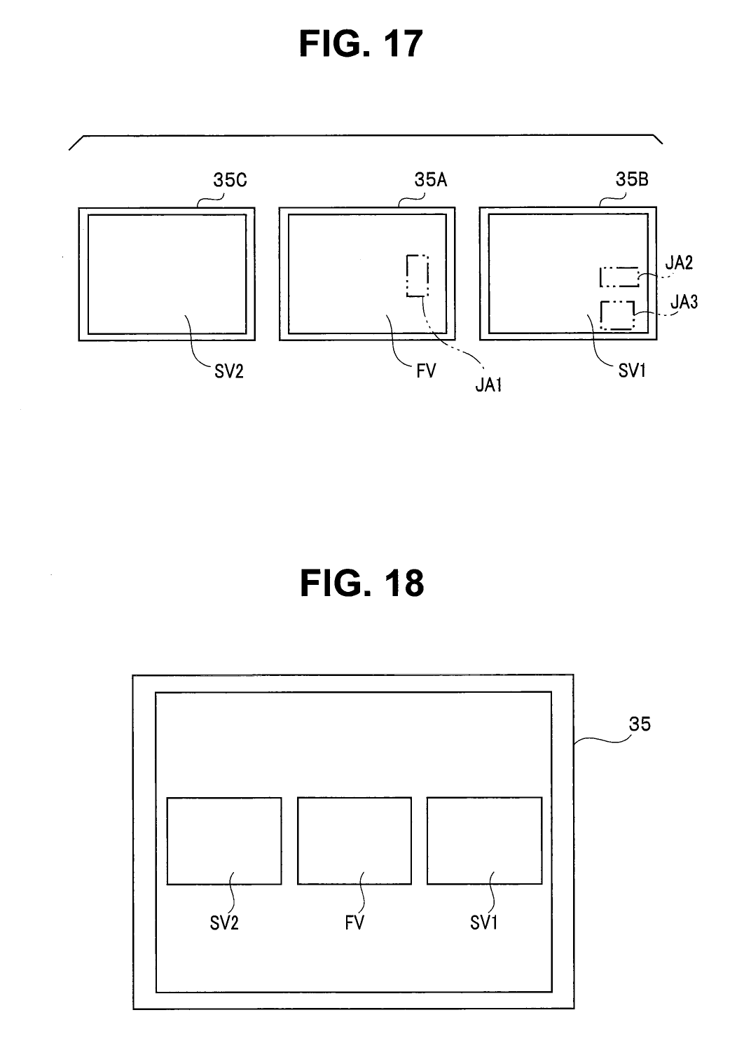

FIG. 17 is a diagram showing a display example of three endoscopic images displayed on the three monitors 35A, 35B and 35C. The forward field-of-view image FV is displayed on the monitor 35A in a center; the right-side lateral field-of-view image SV1 is displayed on the monitor 35B on the right side; and the left-side lateral field-of-view image SV2 is displayed on the monitor 35C on the left side. That is, there are two lateral field-of-view images, and the control portion 45A controls output of the image signal of the forward field-of-view image FV and the image signals of the two lateral field-of-view images SV1 and SV2 so that the forward field-of-view image FV is arranged in the center and is sandwiched between the two lateral field-of-view images SV1 and SV2, among the monitors 35A, 35B and 35C.

Three images acquired at the three observation windows 12A, 13A and 13B are displayed on the monitors 35A, 35B and 35C, respectively.

Similarly to the first embodiment, judgment areas JA1, JA2 and JA3 and a mask area can be set in each image.

Note that it is also possible to display the three endoscopic images side by side on the screen of one monitor 35.

FIG. 18 is a diagram showing a display example of three endoscopic images displayed on one monitor 35. It is also possible to display the forward field-of-view image FV in a central part of the screen of the monitor 35, the right-side lateral field-of-view image SV1 on a right side of the screen of the monitor 35, and the left-side lateral field-of-view image SV2 on a left side of the screen of the monitor 35.

Note that the monitor selection signal MS is used in the case of displaying a plurality of endoscopic images on a plurality of monitors but is not used in the case of displaying a plurality of endoscopic images on one monitor.

As described above, the endoscope 2A of the present invention is capable of acquiring three endoscopic images so that a wide-angle range can be observed, and F. the video processor 32A is capable of displaying the three endoscopic images on the three monitors 35A, 35B and 35C.

Furthermore, the image outputting portion 49A is configured so as to be able to control which of the three monitors 35A, 35B and 35C each of the inputted three endoscopic images is outputted to, based on the monitor selection signal MS from the control portion 45A.

### (Operation)

The endoscope system 1A also has the automatic image display switching mode and is capable of executing each of processes shown in FIGS. 6 to 9 similarly to the endoscope system 1 of the first embodiment.

A flow of a process performed when the endoscope system 1A is set to the automatic image display switching mode by the user is same as the flow of FIG. 6. However, each of settings (S1 and S7) is performed for three endoscopic images, and detection of an image change amount (S3) is performed for each of judgment areas of the three endoscopic images. Display control (S6) is also performed so as to control display states of the three monitors 35A, 35B and 35C.

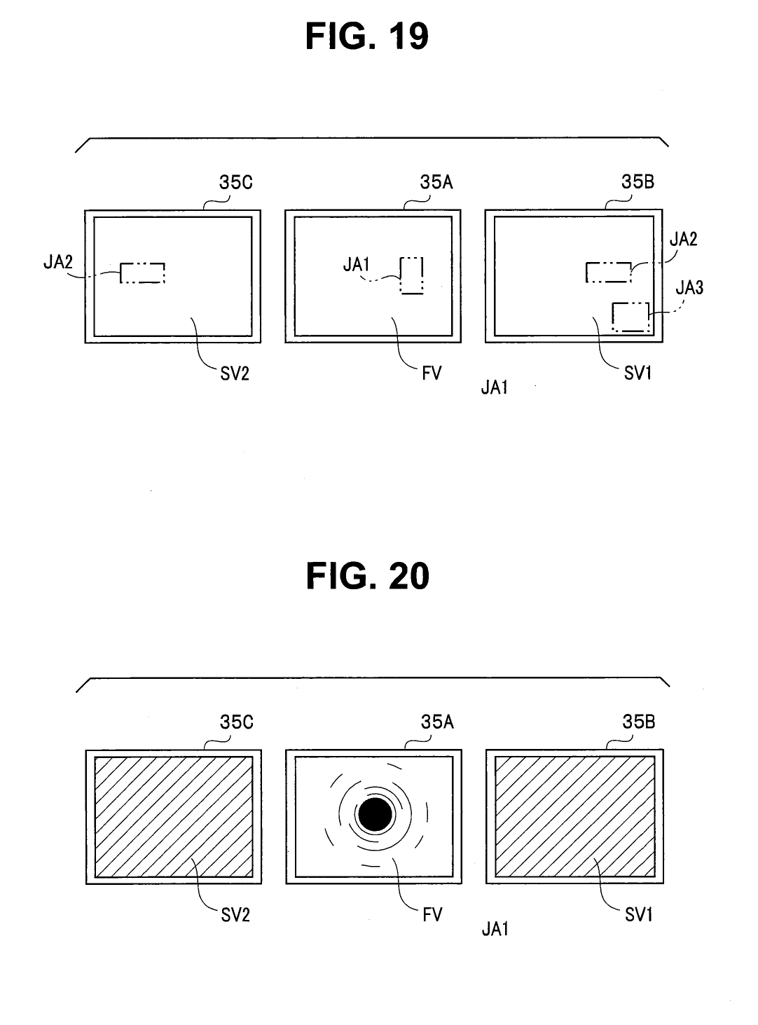

FIG. 19 is a diagram showing an example of observation images displayed on the three monitors 35A, 35B and 35C when the endoscope system 1A is set to the automatic image display switching mode.

As shown in FIG. 19, immediately after the automatic image display switching mode is set, the forward field-of-view image FV, the lateral field-of-view image SV1 of a right-side field of view and the lateral field-of-view image SV2 of a left-side field of view are displayed on the three monitors 35A, 35B and 35C, respectively.

When a state of use of the user is judged to be the insertion state, the control portion 45A performs display control so that observation images displayed on the three monitors 35A, 35B and 35C are to be changed into an observation image display state corresponding to the insertion state, for example, as shown in FIG. 20.

FIG. 20 is a diagram showing an example of the observation images displayed on the three monitors 35A, 35B and 35C in the insertion state.

In the insertion state, since an image which the user is mainly interested in is the forward field-of-view image FV, display of the monitors 35A, 35B and 35C is controlled so that the forward field-of-view image FV is displayed on the monitor 35A, and the two lateral field-of-view image SV1 and SV2 are not displayed on the monitors 35B and 35C (indicated by oblique lines).

In the case of FIG. 20, the control portion 45A outputs a monitor selection signal MS for displaying the forward field-of-view image FV on the monitor 35A and displaying nothing on the monitors 35B and 35C, to the image outputting portion 49A.

Note that, instead of displaying nothing on the monitors 35A and 35B, a masking process for covering display may be performed on a part or whole of portions of the monitors 35A and 35B where the lateral field-of-view image SV1 and SV2 are displayed.

When the state of use of the user is judged to be the screening state, the control portion 45A performs display control so as to change the observation image display state, for example, to an observation image display state as shown in FIG. 19.

In the screening state, since the user is mainly interested in not only the forward field-of-view image FV but also the two lateral field-of-view image SV1 and SV2, display of the monitors 35A, 35B and 35C is controlled so as to display the forward field-of-view image FV on the monitor 35A and display the two lateral field-of-view images SV1 and SV2 on the monitors 35B and 35C.

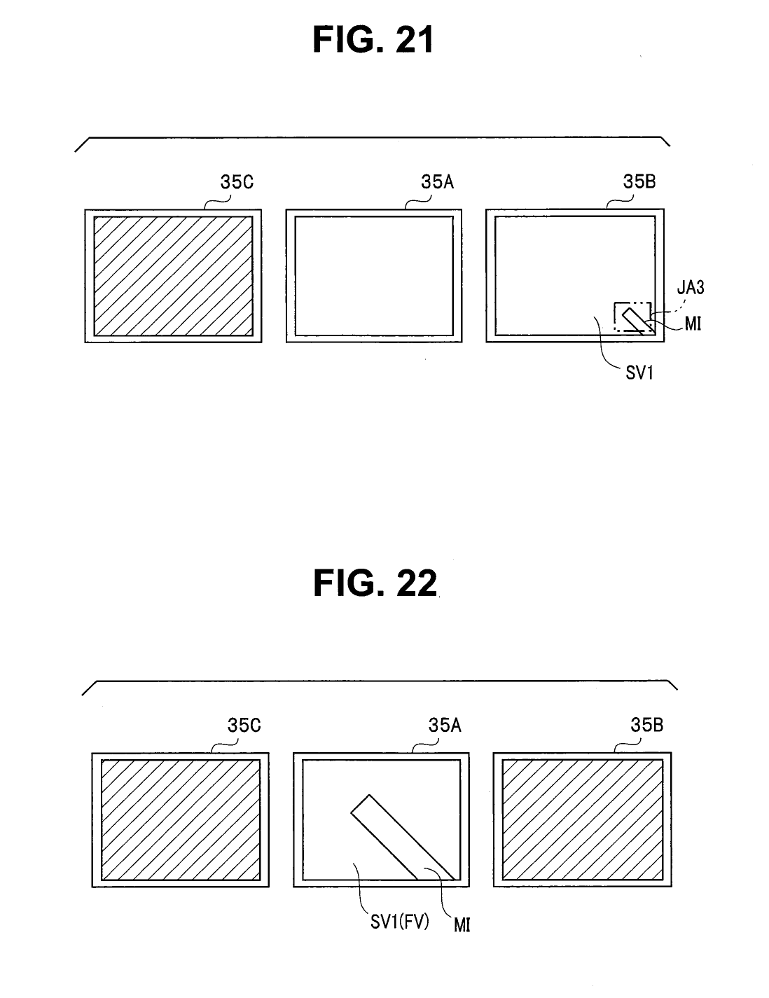

When the state of use of the user is judged to be the treatment instrument used state, the control portion 45A performs display control so as to change the observation display state to an observation image display state that the lateral field-of-view image SV1 in which the treatment instrument MI is displayed is displayed on the monitor 35B among the three monitors 35A, 35B and 35C, for example, as shown in FIG. 21.

FIG. 21 is a diagram showing an example of observation images displayed on the three monitors 35A, 35B and 35C in the treatment instrument used state.

In the treatment instrument used state, since an image which the user is mainly interested in is the lateral field-of-view image SV1 in which the treatment instrument MI is displayed, display of the monitors 35B, 35A and 35C is controlled so that the lateral field-of-view image SV1 is displayed on the monitor 35B, and the lateral field-of-view image SV2 is not displayed on the monitors 35A or 35C (indicated by oblique lines).

In the case of FIG. 21, the control portion 45A outputs a monitor selection signal MS for displaying the lateral field-of-view image SV1 on the monitor 35B and displaying nothing on the monitors 35C, to the image outputting portion 49A. Further, an area of the treatment instrument MI may be enlarged and displayed on the monitor 35B.

FIG. 22 is a diagram showing an example of an observation image displayed on the monitor 35A by enlarging the image area which includes the treatment instrument MI in the lateral field-of-view image SV1.

An image which the user is mainly interest in is the lateral field-of-view image SV1 in which the treatment instrument MI is displayed. Therefore, as shown in FIG. 22, the control portion 45A enlarges a part of the lateral field-of-view image SV1, that is, the image area which includes the treatment instrument MI and displays the image area on the monitor 35A in the center which the use can easily see.

In the case of FIG. 22, the control portion 45A outputs a control signal EC1 for enlarging the area in the lateral field-of-view image SV1 which includes the treatment instrument MI to the enlarging/reducing circuit 43A, and outputs a monitor selection signal MS for displaying a partially enlarged image of the lateral field-of-view image SV1 on the monitor 35A and displaying nothing on the monitor 35B, to the image outputting portion 49A.

Note that it is preferable to display the forward field-of-view image FV on the monitor 35C so that it is easy for the user to continuously confirm the forward direction.

Furthermore, when a tip of the treatment instrument MI projects and is displayed in the forward field-of-view image FV, the forward field-of-view image FV which includes the treatment instrument MI is displayed on the monitor 35A as shown in FIG. 22.

Further, in the suction state, the control portion 45A causes the display state, for example, to be a display state as shown in FIG. 20. In the present embodiment also, it may be added to judgment conditions that, if liquid is detected in the forward field-of-view image FV by image processing, the suction operation button 26 has been operated.

Note that, in order to, even if a state of not displaying a secondary image on a monitor continues, cause the secondary image to be displayed again when the use state of the endoscope 2A changes, the video processor 32A continues detecting amounts of change in a primary image and the secondary image though the secondary image is not displayed in the present embodiment.

Though the mechanism realizing the function of illuminating and observing the lateral direction is included in the insertion portion 4 together with the mechanism realizing the function of illuminating and observing the forward direction, the mechanism realizing the function of illuminating and observing the lateral direction may be a separate body attachable to and detachable from the insertion portion 4.

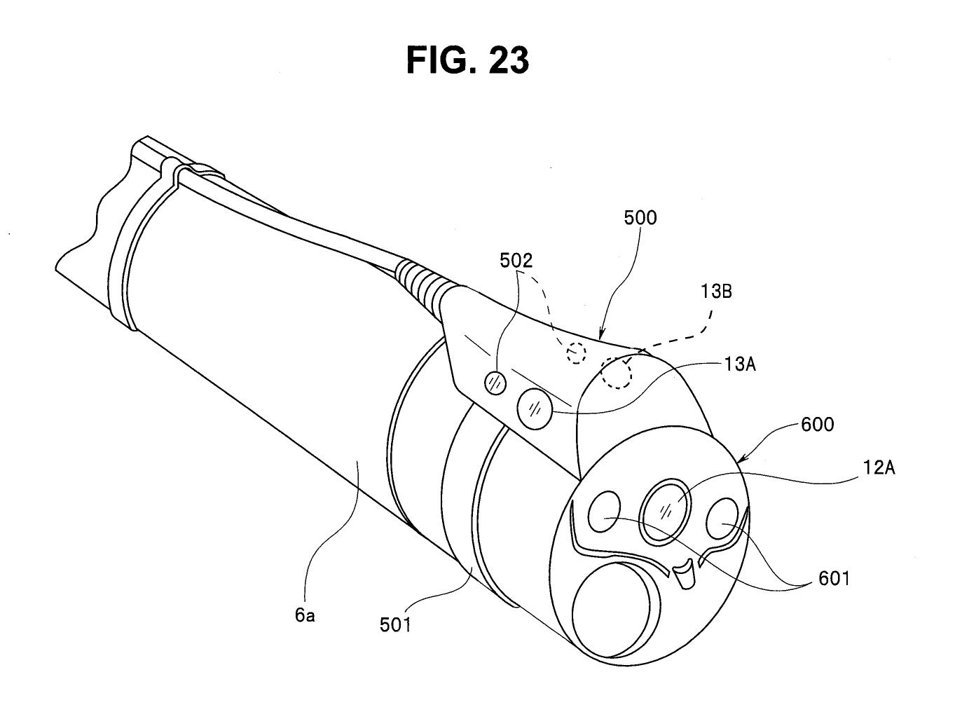

FIG. 23 is a perspective view of a distal end portion 6a of the insertion portion 4 to which a unit for lateral observation is attached, according to a modification of the second embodiment. The distal end portion 6a of the insertion portion 4 has a unit for forward field of view 600. A unit for lateral field of view 500 is configured to be attachable to and detachable from the unit for forward field of view 600 with a clip portion 501.

The unit for forward field of view 600 has a forward observation window 12A for acquiring a forward field-of-view image FV and an illumination window 601 for illuminating the forward direction. The unit for lateral field of view 500 has two lateral observation windows 13A and 13B for acquiring images in left and right directions and two illumination windows 502 for illuminating the left and right directions.

A video processor 32A and the like can acquire and display an observation image as shown in the embodiment described above by performing lighting and extinction of each illumination window 502 of the unit for lateral field of view 500 according to a frame rate of the forward field of view.

As described above, according to the embodiment described above, it is possible to provide an endoscope system capable of displaying an observation image an information amount of which has been controlled to be an optimum information amount required, according to a use state of an endoscope.

Note that, though values of pixels of at least one color among a plurality of color pixels of an image signal in a predetermined area or a magnitude of an edge component of the image signal is used to detect an amount of change in an image in the two embodiments described above, colors and saturations calculated from the color pixels or a luminance value of each pixel of an image signal in a predetermined area may be used.

Though an endoscope configured to display a wide-angle field of view has been described as an example in the above embodiments, the spirit of the present invention may be applied to a side-view endoscope. In that case, a primary image is a lateral field-of-view image, and a secondary image is a forward field-of-view image, for example, for confirming an insertion direction at time of insertion up to an appropriate part.

The present invention is not limited to the embodiments described above, and various kinds of changes, alterations and the like are possible within a range not departing from the spirit of the present invention.

What is claimed is:

1. An endoscope system comprising:
an insertion portion configured to be inserted into an inside of an object;
a first object image acquiring portion provided on the insertion portion and configured to acquire a first object image from a first area of the object;
a second object image acquiring portion provided on the insertion portion and configured to acquire a second object image from a second area of the object different from the first area;
an image change amount detecting portion configured to detect an amount of change in an image signal in a predetermined area of at least one of the first object image and the second object image within a predetermined time period; and
an image signal generating portion configured to generate an image signal based on the first object image and generate an image signal obtained by changing a display information amount of the second object image according to the amount of the change in the image signal detected by the image change amount detecting portion.

2. The endoscope system according to claim 1, wherein the image change amount detecting portion detects the amount of the change in the image signal of the predetermined area in at least one of the first object image and the second object image in a state of being displayed on a display portion within the predetermined time period.

3. The endoscope system according to claim 1, wherein the image change amount detecting portion detects the amount of the change in the image signal of the predetermined area in at least one of the first object image and the second object image in a state of not being displayed on the display portion within the predetermined time period.

4. The endoscope system according to claim 1, wherein the image change amount detecting portion detects the amount of the change in the image signal in the predetermined area based on at least one of a luminance value of each pixel, a pixel value, color and saturation of at least one of a plurality of color pixels, and a magnitude of an edge component.

5. The endoscope system according to claim 1, further comprising a use state judging portion configured to judge which one is a use state among: a state of an insertion operation of the insertion portion being performed, a screening state in which an operation speed is slower than an operation speed of the insertion operation, a state of using a treatment instrument from a distal end of the insertion portion, and a state of suction in the object, based on a result of the detection by the image change amount detecting portion.

6. The endoscope system according to claim 5, wherein the use state judging portion judges the use state of the insertion portion based on a detection result obtained by weighting the amount of the change detected by the image change amount detecting portion.

7. The endoscope system according to claim 1, wherein
the first object image is an object image of the first area that includes a forward direction of the insertion portion substantially parallel to a longitudinal direction of the insertion portion; and
the second object image is an object image of the second area that includes a lateral direction of the insertion portion in a direction crossing a longitudinal direction of the insertion portion.

8. The endoscope system according to claim 1, further comprising an image pickup portion configured to photoelectrically convert the first object image acquired by the first object image acquiring portion and the second object image acquired by the second object image acquiring portion, on one image pickup surface.

9. The endoscope system according to claim 1, wherein
the first object image acquiring portion is arranged on a distal end portion of the insertion portion in a longitudinal direction so as to acquire the first object image from the first area that is in a direction in which the insertion portion is inserted; and
the second object image acquiring portion is arranged along a circumferential direction of the insertion portion so as to acquire the second object image from the second area.

10. The endoscope system according to claim 9, wherein
the first object image is in a substantially circular shape; and
the second object image is in a substantially annular shape surrounding at least a part of a circumference of the first object image.

11. The endoscope system according to claim 1, wherein the image signal generating portion performs any one of a masking process, an enlargement/reduction process and a hiding process for a display portion, for the second object image according to the amount of the change in the image signal detected by the image change amount detecting portion.

12. The endoscope system according to claim 1, wherein the image signal generating portion generates an image signal in which the second object image is arranged so as to be next to the first object image.

13. The endoscope system according to claim 12, wherein
the second object image includes object images acquired from two directions; and
the image signal generating portion generates an image signal in which the first object image is arranged in a center, and the second object image acquired from the two directions is arranged so as to sandwich the first object image.

14. The endoscope system according to claim 13, further comprising two image pickup portions for acquiring the second object image acquired from the two directions; wherein
the two image pickup portions are arranged at substantially equal angles in a circumferential direction of the insertion portion.

15. The endoscope system according to claim 1, comprising:
a first image pickup portion configured to pick up the first object image; and
a second image pickup portion configured to pick up the second object image, the second image pickup portion being different from the first image pickup portion; wherein
an image signal of the first object image is generated from an image obtained by the first image pickup portion; and
an image signal of the second object image is generated from an image obtained by the second image pickup portion.
